SEQUENCE LISTING (24 sequences)

SEQ ID NO: 1 - protein, 166 aa, Homo sapiens
  SOURCE 1..166: /mol_type=“protein”/organism=“Homo sapiens”
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 2 - protein, 49 aa, Homo sapiens
  SOURCE 1..49: /mol_type=“protein”/organism=“Homo sapiens”
ILAFQLCIVLGSLGCYCQDPYVKEAENLKKYFNAGHSDVADNGTLFLGI

SEQ ID NO: 3 - protein, 143 aa, Homo sapiens
  SOURCE 1..143: /mol_type=“protein”/organism=“Homo sapiens”
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 4 - protein, 143 aa, Homo sapiens
  SOURCE 1..143: /mol_type=“protein”/organism=“Homo sapiens”
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 5 - protein, 55 aa, Homo sapiens
  SOURCE 1..55: /mol_type=“protein”/organism=“Homo sapiens”
QSQIVSFYFKLFKNFKDDQSIQKSVETIKEDMNVKFFNSNKKKRDDFEKLTNYSV

SEQ ID NO: 6 - protein, 46 aa, Homo sapiens
  SOURCE 1..46: /mol_type=“protein”/organism=“Homo sapiens”
MNVKFFNSNKKKRDDFEKLTNYSVTDLNVQRKAIHELIQVMAELSP

SEQ ID NO: 7 - protein, 39 aa, Homo sapiens
  SOURCE 1..39: /mol_type=“protein”/organism=“Homo sapiens”
SVETIKEDMNVKFFNSNKKKRDDFEKLTNYSVTDLNVQR

SEQ ID NO: 8 - protein, 25 aa, Homo sapiens
  SOURCE 1..25: /mol_type=“protein”/organism=“Homo sapiens”
VTDLNVQRKAIHELIQVMAELSPAA

SEQ ID NO: 9 - protein, 144 aa, artificial sequence
  SOURCE 1..144: /mol_type=“protein”/note=“recombinant human IFN gamma”/organism=“artificial sequences”
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 10 - protein, 41 aa, Homo sapiens
  SOURCE 1..41: /mol_type=“protein”/organism=“Homo sapiens”
SYILAFQLCIVLGSLGCYCQDPYVKEAENLKKYFNAGHSDV

SEQ ID NO: 11 - protein, 21 aa, Homo sapiens
  SOURCE 1..21: /mol_type=“protein”/organism=“Homo sapiens”
ETIKEDMNVKFFNSNKKKRDD

SEQ ID NO: 12 - protein, 144 aa, artificial sequence [sequence omitted]

SEQ ID NO: 13 - protein, 458 aa, Homo sapiens
  SOURCE 1..458: /mol_type=“protein”/organism=“Homo sapiens”
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 14 - protein, 434 aa, Homo sapiens
  SOURCE 1..434: /mol_type=“protein”/organism=“Homo sapiens”
GKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 15 - protein, 47 aa, Homo sapiens
  SOURCE 1..47: /mol_type=“protein”/organism=“Homo sapiens”
IGLGIFFCVRCRHRRRQAERMSQIKRLLSEKKTCQCPHRFQKTCSPI

SEQ ID NO: 16 - protein, 36 aa, Homo sapiens
  SOURCE 1..36: /mol_type=“protein”/organism=“Homo sapiens”
GKKVVLGKKGDTVELTCTASQKKSIQFHWKNSNQIK

SEQ ID NO: 17 - protein, 15 aa, Homo sapiens
  SOURCE 1..15: /mol_type=“protein”/organism=“Homo sapiens”
DTYICEVEDQKEEVQ

SEQ ID NO: 18 - protein, 25 aa, Homo sapiens
  SOURCE 1..25: /mol_type=“protein”/organism=“Homo sapiens”
KEEVQLLVFGLTANSDTHLLQGQSL

SEQ ID NO: 19 - protein, 235 aa, Homo sapiens
  SOURCE 1..235: /mol_type=“protein”/organism=“Homo sapiens”
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 20 - protein, 20 aa, Homo sapiens
  SOURCE 1..20: /mol_type=“protein”/organism=“Homo sapiens”
PLALLLHAARPSQFRVSPLD

SEQ ID NO: 21 - protein, 20 aa, Homo sapiens
  SOURCE 1..20: /mol_type=“protein”/organism=“Homo sapiens”
AEGLDTQRFSGKRLGDTFVL

SEQ ID NO: 22 - protein, 20 aa, Homo sapiens
  SOURCE 1..20: /mol_type=“protein”/organism=“Homo sapiens”
SIMYFSHFVPVFLPAKPTTT

SEQ ID NO: 23 - protein, 10 aa, Homo sapiens
  SOURCE 1..10: /mol_type=“protein”/organism=“Homo sapiens”
VITLYCNHRN

SEQ ID NO: 24 - protein, 15 aa, Homo sapiens
  SOURCE 1..15: /mol_type=“protein”/organism=“Homo sapiens”
VVKSGDKPSLSARYV

(19) United States
(12) Patent Application Publication
(10) Pub. No.: US 2013/0171161 A1
(43) Pub. Date: Jul. 4, 2013

(54) METHOD AND COMPOSITION FOR THE TREATMENT OF DISEASES CAUSED BY OR ASSOCIATED WITH HIV

(71) Applicants: Oleg Iliich Epshtein, Moscow (RU); Andrey Valerievich Strygin, Volgograd (RU)

(72) Inventors: Oleg Iliich Epshtein, Moscow (RU); Andrey Valerievich Strygin, Volgograd (RU)

(73) Assignee: Oleg Iliich Epshtein, Moscow (RU)

(21) Appl. No.: 13/336,775

(22) Filed: Dec. 23, 2011

Related U.S. Application Data
(60) Provisional application No. 61/426,802, filed on Dec. 23, 2010.

(51) Int. Cl. A61K 39/395; A61K 31/506; A61K 45/00

(52) U.S. Cl. CPC A61K 39/3955; A61K 45/00; A61K 31/506
USPC 424/145.1; 424/158.1

(57) ABSTRACT

The invention provides a combination pharmaceutical composition comprising a) at least one activated-potentiated form of an antibody to at least one cytokine or at least an activated-potentiated form of an antibody to at least one receptor; and b) an effective amount of a nucleoside reverse transcriptase inhibitor, wherein said at least one cytokine or at least one receptor is participating in the regulation of immune process. Various embodiments and variants are contemplated.

The invention also provides a method of treatment or prophylaxis of HIV, including AIDS, which includes administration of the combination pharmaceutical composition described in the specification to the patient in need thereof.

### RELATED APPLICATIONS

The present application claims priority from U.S. Provisional Application No. 61/426,802, filed on Dec. 23, 2010, the entire disclosure and content of which is incorporated by reference in its entirety.

### FIELD OF THE INVENTION

The invention relates the treatment of diseases caused by or associated with HIV, inclusive of AIDS.

### BACKGROUND OF THE INVENTION

The availability of the antiretroviral therapy (ARVT) improved the prognosis for a considerable number of HIV infected patients. [Navin T. R. 2000; Breitstein P., 2006; Price P., 2001]. Various ARVT drugs have been approved for treatment of HIV infection. For example, zidovudine (AZT or Retrovir®) a nucleoside reverse transcriptase inhibitor or NRTI which interfere with the virus's nucleotide sequencing.

By suppressing HIV replication, ARVT allows regeneration of the immune system [Abubakar I., 2007; Batteagy M., 2006], thereby reducing the risk of opportunistic infections (which have been defined as infections that are more frequent or more severe because of immunosuppression in HIV-infected persons) and neoplasms [Egger. M., 2002; Navin T. R. 2000]. However, the risk of the opportunistic infection is rather high, especially within few first months of the treatment [Lawn S. D., 2008]. It was noted that the introduction of ARVT treatment did not produce any changes in the frequency of infection caused by human papilloma virus, resulting in neoplasms and hepatitis C virus [Vetga A. P., 2006]. Further, while ARVT was felt to be very promising in the earlier stages, development of resistance to them has caused a considerable amount of disappointment and frustration. Thus, despite the availability of ARVT in the United States and other industrialized countries, opportunistic infections remained the main reason of mortality among HIV-infected patients.

Highly active antiretroviral therapy (HAART) has proven to be an effective method of preventing opportunistic infections, leading to changes in the guidelines and recommendations for primary and secondary prophylaxis for such opportunistic infections [Hermsen E. D., 2004]. However, due to a wide range of side effects, in the majority of cases, primary and secondary specific opportunistic preventive measures had to be ceased [Dworkin M. S., 2000]. In addition, the high dosages needed increased the potential for toxicity, product interaction, resistance to microorganisms as well as the price of treatment [Furrer H., 1999; Green H., 2004; Lopez Bemaldo de Quiros J. C., 2001; Mussini C., 2000] which also leads to noncompliance. Thus, the complete elimination of opportunistic infections is still an unattainable goal and the spectrum and relative risk of the opportunistic infections has not changed. For example, Pneumocystic pneumonia (Pneumocystis jiroveci) and Mycobacterium avium infection are still wide spread [Kaplan I., 2000]. Therefore, there is still a need for preventing opportunistic infections among HIV-infected patients [Hermsen E. D., 2004].

The therapeutic effect of an extremely diluted form (or ultra-low form) of antibodies potentized by homeopathic technology (activated-potentiated form) has been discovered by Dr. Oleg I. Epshtein. For example, U.S. Pat. No. 7,582,294 discloses a medicament for treating Benign Prostatic Hyperplasia or prostatitis by administration of a homeopathically activated form of antibodies to prostate specific antigen (PSA). Ultra-low doses of antibodies to gamma interferon have been shown to be useful in the treatment and prophylaxis of treating diseases of viral etiology. See U.S. Pat. No. 7,572,441, which is incorporated herein by reference in its entirety.

Treatment of viral diseases based on ultra-low doses of antibodies to interferon is known in the art (U.S. Pat. No. 7,572,441, Aug. 11, 2009). Anaferon is a preparation containing antibodies to human IFN-γ, which is registered as a medical product (antiviral and immunemodullating) by the Federal Service for the Supervision of Public Health and Social Development (Roszdravnadzor) in 2009. Anaferon influences the immunoregulation processes, enhances the activation of the antiviral immune system and stimulates the production of the main cytotoxicants. As a result it possesses the prospective of being used within the framework of combined therapy in HIV infected patients as a prophylactic drug and preparation for treatment of acute respiratory diseases (ARD) and opportunistic infections in HIV infected patients, as well as a preparation, enhancing the ARVT efficiency [Sherstoboev E. Y., 2003; Lytkina I. N., 2004; Revalina V. A., 2006].

### SUMMARY OF THE INVENTION

The present invention provides a pharmaceutical composition comprising (i) a nucleoside reverse transcriptase inhibitor and (ii) an activated (potentiated) antibodies to an antigen, wherein the antigen is a protein or peptide (polypeptide) of the immune system (or, predominantly, produced by the immune system) which interact with HIV or content and/or fictional activity of which is changed due to the HIV infection.

Preferably the nucleoside reverse transcriptase inhibitor is azidothymidine injection.

The antigen may be receptors connected or not connected to the external membrane cells of the immune system. Preferably the antigen may be a glycoprotein expressed on the surface, for example cluster of differentiation, preferably CD4 and CD8. The antigen may also be IFN-gamma.

Preferably, the pharmaceutical composition including said activated-potentiated form of an antibody is in the form of a mixture of C12, C30, and C200 homeopathic dilutions. It is specifically contemplated that said mixture of C12, C30, and C200 homeopathic dilutions is impregnated onto a solid carrier.

The activated-potentiated form of an antibody may be a monoclonal, polyclonal or natural antibody. It is specifically contemplated that the activated-potentiated form of an antibody is a polyclonal antibody. The invention provides activated-potentiated forms of antibodies to antigen(s) having sequences described in the specification and claimed in the appended claims.

In a variant, the pharmaceutical composition includes activated-potentiated form of an antibody prepared by successive centesimal dilutions coupled with shaking of every dilution. Vertical shaking is specifically contemplated.

In another aspect, the invention provides a method of treating and preventing the diseases caused by HIV or associated with HIV, said method comprising administering to a patient in need thereof (i) a nucleoside reverse transcriptase inhibitor and (ii) an activated (potentiated) antibodies to a protein or peptide (polypeptide) of the immune system (or, predominantly, produced by the immune system) which interact with HIV or content and/or fictional activity of which is changed due to the HIV infection.

In an embodiment, the pharmaceutical composition is administered in the form of a solid oral dosage form which comprises a pharmaceutically acceptable carrier and said nucleoside reverse transcriptase inhibitor and activated-potentiated form of an antibody impregnated onto said carrier. In a variant, said solid oral dosage form is a tablet. Variants and embodiments are provided.

In accordance with the method aspect of the invention, the pharmaceutical composition may be administered in one to two unit dosage forms, each of the dosage form being administered from once daily to four times daily. In a variant, the pharmaceutical composition is administered twice daily, each administration consisting of two oral dosage forms. In a variant, the pharmaceutical composition is administered in one to two unit dosage forms, each of the dosage forms being administered twice daily. All variants and embodiments described with respect to the composition aspect of the invention may be used with the method aspect of the invention.

### BRIEF DESCRIPTION OF THE FIGURES

FIG. 1. Shows the dynamics of changes in CD3 cells amount in the peripheral blood within the use of “Anaferon”.

FIG. 2. Shows the dynamics of changes in CD4 cells amount in the peripheral blood within the use of “Anaferon”.

FIG. 3. Shows the dynamics of changes in CD4 cells amount in the peripheral blood within the use of “Anaferon”.

FIG. 4. Shows the dynamics of changes in CD4/CD8 balance in the peripheral blood within the use of “Anaferon”.

FIG. 5. Shows the content of interleukin-2 in the patients' blood serum in dynamics within the groups under research (prior and subsequent to “Anaferon” course).

FIG. 6. Shows the content of interleukin-4 in the patients' blood serum in dynamics within the groups under research (prior and subsequent to “Anaferon” course).

FIG. 7. Shows the content of gamma interferon in the patients' blood serum in dynamics within the groups under research (prior and subsequent to “Anaferon” course).

FIG. 8. Shows the IFN-γ/IL-4 interrelation in dynamics within the groups under research (prior and subsequent to “Anaferon” course).

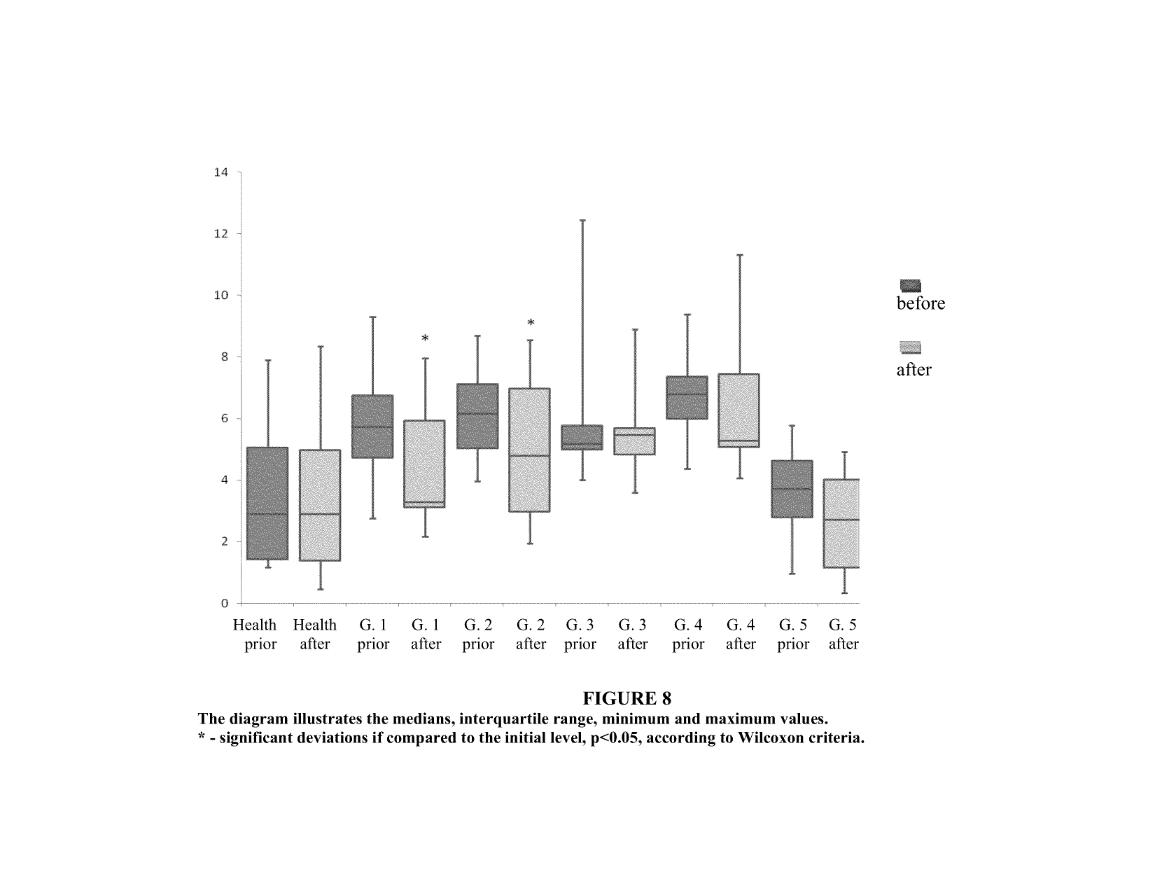

### DETAILED DESCRIPTION OF THE INVENTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)₂, Fab′, and the like. The singular “antibody” includes plural “antibodies.”

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³⁰ and 100⁵⁰ times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term ‘activated-potentiated” form used in the claims.

In other words, an antibody is in the “activated-potentiated” or “potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well-accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated-potentiated” form of the antibodies described herein is described in more detail below.

There has been a considerable amount of controversy regarding homeopathic treatment of human subjects. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models. Human clinical studies also provide evidence that the activity observed in the animal model is well translated to human therapy. Human studies have also provided evidence of availability of the “activated potentiated” forms described herein to treat specified human diseases or disorders well accepted as pathological conditions in the medical science; it is associated with higher antiviral and, possibly, immunotropic action, intensification of activation of CD4 lymphocytes and enrichment of number of receptors on the surface of CD4 cells.

Thus, loss of viral load is observed as a result of repression of HIV entering the cells (exhibited as a change in functional activity of CD4 receptors through which HIV enters the cells); repression of replication of HIV inside the cells, activation of the process of transcription of mRNK of antiviral protein (protein kinase PKR, oligoadenylate synthetase, adenozime deaminase), Mx, MHC I and II protein etc.). Thus, the claimed medicinal product possesses high preventive effectiveness with respect to HIV, preventing infection of the cells by HIV and its endocellular replication. It can be used either for effective treatment or for preventive measures of chronic viral diseases, including secondary prevention of HIV infection.

Also, the claimed “activated-potentiated” form of antibody encompasses only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated’ form of the antibodies of the present invention is not attributable to the initial molecular form of the antibody. Preferred is the “activated-potentiated” form of antibody in liquid or solid form in which the concentration of the molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the molecular form of the antibody is below the Avogadro number. In the pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

The present invention provides a pharmaceutical composition that includes two active ingredient components: a) at least one activated-potentiated form of an antibody to at least one cytokine or at least an activated-potentiated form of an antibody to at least one receptor; and b) an effective amount of a nucleoside reverse transcriptase inhibitor, wherein said at least one cytokine or at least one receptor is participating in the regulation of immune process.

One active ingredient component is nucleoside reverse transcriptase inhibitor. The preferred nucleoside reverse transcriptase inhibitor is azidothymidine (AZT). AZT is 1[2R,4S,5S)-4-azido-5-(hydroxymethyl)oxolan-2-yl]-5-methylpyrimidine-2,4-dione. AZT was approved in the United States for treatment of AIDS in 1987. AZT was approved in the United States as a preventative treatment in 1990. AZT's usual dose is 600 mg per day. In the combination pharmaceutical composition of the present invention, it is contemplated that AZT may be included in the HIV treatment or preventative regimen in the same effective dose as currently used for mono-administration of AZT or for administration of AZT in combination with other known antiretroviral drugs. Chronic, high-dose administration of AZT has been associated with significant side effects, including anemia, neutropenia, hepatoxicity and others. See, e.g., Sun. R et al, Identification and Characterization of Mitochondrial factors Modulating Thymidine Kinase 2 Activity, Nucleosids, Nucleotides and Nucleic Acids, 29 (4-6): 382-385 (2010); Scruggs et al, Mechanisms of zidovudine-induced Mitochondrial Toxicity and Myopathy, Pharmacology, 82 (2): 83-88 (2008). Therefore, in the combination pharmaceutical composition of the present invention, it is also contemplated that AZT may be included in the HIV treatment or preventative regimen at lower effective dose than with mono-administration of AZT or with administration of AZT with other known antiretroviral drugs. The magnitude of a prophylactic or therapeutic dose of the nucleoside reverse transcriptase inhibitor, preferably AZT, in the acute or chronic management of a disease will vary with the severity of the condition to be treated and the route of administration. The dose, and perhaps the dose frequency, will also vary according to age, body weight, response, and the past medical history of the individual patient. In general, the recommended daily dose range for the conditions described herein lie within the range of from about 200 mg to about 600 mg per day. Preferably, a daily dose range should be from about 300 mg to about 600 mg per day, more preferably, between about 300 mg and about 400 mg per day. The contemplated pharmaceutical composition of the present invention may, for example, include dosage forms containing nucleoside reverse transcriptase inhibitor, preferably AZT, in the amount of 100 mg, 200 mg, and 300 mg. The reduction in the antiviral therapeutic dose is believed to lower the risk of rapid resistance development and increases the ability to use nucleoside reverse transcriptase inhibitors, and particularly AZT, for long-term preventive use and treatment of the HIV infection. The pharmaceutical composition described herein expands the arsenal of the medical preparations available for treatment and prophylaxis of HIV infection. The preparation and use of AZT is disclosed in U.S. Pat. Nos. 4,724,232, 4,818,538, 4,828,838, 4,833,130 and 4,837,208, all of which are incorporated by reference in their entirety and for the purposes stated.

Another active ingredient component is at least one activated-potentiated form of an antibody to at least one cytokine or at least an activated-potentiated form of an antibody to at least one receptor. The preparation, use and properties of suitable activated-potentiated forms are described in U.S. application Ser. No. 13/135,899 and PCT/RU20011/000523, which are hereby incorporated by reference for the purposes stated and in their entirety. The preferred activated-potentiated form is the activated-potentiated form of antibody to gamma-interferon, the preparation, use, and properties of which is described in U.S. Pat. No. 7,572,441 and U.S. Pat. No. 8,066,992, which are incorporated by reference for the purpose stated.

The activated-potentiated form of antibodies to IFN-gamma, or activated (potentiated) form of antibodies to CD4 or activated (potentiated) form of antibodies to CD8 prepared according to the homeopathic technology of potentiation by repeated, consistent dilution and intermediate external action of shaking as described in more detail herein below administered simultaneously with a nucleoside reverse transcriptase inhibitor, preferably AZT. The pharmaceutical composition of the invention is particularly useful in the treatment and prophylaxis of the diseases caused by HIV or associated with HIV, including AIDS. As shown in the Examples, the pharmaceutical composition of the invention possesses unexpected synergetic therapeutic effect, which manifest itself in particular therapeutic effectiveness in treatment of diseases caused by HIV or associated with HIV.

The pharmaceutical composition of the invention expands the arsenal of preparations available for the treatment prophylaxis of the diseases caused by HIV or associated with HIV, including AIDS.

The pharmaceutical composition in accordance with this aspect of the invention may be in the liquid form or in solid form. Activated potentiated form of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R. —2005—Vol. 14. —N 1-2. P.33-55, both incorporated herein by reference.

Monoclonal antibodies may be obtained, e.g., by means of hybridoma technology. The initial stage of the process includes immunization based on the principles already developed in the course of polyclonal antisera preparation.

Further stages of work involve the production of hybrid cells generating clones of antibodies with identical specificity. Their separate isolation is performed using the same methods as in the case of polyclonal antisera preparation.

Polyclonal antibodies may be obtained via active immunization of animals. For this purpose, for example, suitable animals (e.g. rabbits) receive a series of injections of the appropriate antigen. The animals' immune system generates corresponding antibodies, which are collected from the animals in a known manner. This procedure enables preparation of a monospecific antibody-rich serum.

If desired, the serum containing antibodies may be purified, for example by using affine chromatography, fractionation by salt precipitation, or ion-exchange chromatography. The resulting purified, antibody-enriched serum may be used as a starting material for the preparation of the activated-potentiated form of the antibodies. The preferred concentration of the resulting initial solution of antibody in the solvent, preferably water or a water-ethyl alcohol mixture, ranges from about 0.5 to about 5.0 mg/ml.

The preferred procedure for preparing each component of the combination drug according to the present invention is the use of the mixture of three aqueous-alcohol dilutions of the primary matrix solution of antibodies diluted 100¹², 100³⁰ and 100⁵⁰ times, respectively, which is equivalent to centesimal homeopathic dilutions C12, C30, and C50 or diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In a preferred embodiment, the starting material for the preparation of the activated potentiated form that comprises the pharmaceutical composition of the invention is polyclonal, animal-raised antibody to the corresponding antigen, namely, IFN-gamma or CD4 or activated (potentiated) form of antibodies to CD8. To obtain the activated-potentiated form of polyclonal antibodies to IFN-gamma or CD4 or activated (potentiated) form of antibodies to CD8, the desired antigen may be injected as immunogen into a laboratory animal, preferably, rabbits. Polyclonal antibodies to IFN-gamma may be obtained using the whole molecule of human IFN-gamma of the following sequence:

```
SEQ. ID. NO. 1
Met Lys Tyr Thr Ser Tyr Ile Leu Ala Phe Gln Leu Cys Ile Val
  1 5 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
```

For obtaining of polyclonal antibodies to the human IFN-gamma in the quality of immunogen (antigen) for the rabbit immunization it is also possible use of the polypeptide fragment of the human IFN-gamma with the chain as follows:

```
SEQ. ID. NO. 2
                                                Ile Leu Ala Phe Gln Leu Cys Ile Val
                                                  7 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
Ala Asp Asn Gly Thr Leu Phe Leu Gly Ile
  46 50 55
SEQ. ID. NO. 3
                                                                Gln Asp Pro Tyr Val Lys Glu
                                                                24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
SEQ. ID. NO. 4
                                                                Gln Asp Pro Tyr Val Lys Glu
                                                                24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Gln Gly Arg Arg Ala Ser
151 155 160 165
Gln
166
SEQ. ID. NO. 5
                                                                Gln Ser Gln Ile Val Ser Phe
                                                                69 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val
121 123
SEQ. ID. NO. 6
                                                                        Met Asn Val Lys Phe Phe
                                                                        100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro
136 140 145
SEQ. ID. NO. 7
        Ser Val Glu Thr Ile Lys Glu Asp Met Asn Val Lys Phe Phe
        92 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg
121 125 130
SEQ. ID. NO. 8
                Val Thr Asp Leu Asn Val Gln Arg Lys Ala Ile His Glu
                123 125 130 135
Leu Ile Gln Val Met Ala Glu Leu Ser Pro Ala Ala
136 140 145 147
SEQ. ID. NO. 9
                                                        Met Gln Asp Pro Tyr Val Lys Glu
                                                                  24 25 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Gln Gly Arg Arg Ala Ser
151 155 160 165
Gln
166
SEQ. ID. NO. 10
                                Ser Tyr Ile Leu Ala Phe Gln Leu Cys Ile Val
                                  5 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
SEQ. ID. NO. 11
                        Glu Thr Ile Lys Glu Asp Met Asn Val Lys Phe Phe
                        94 100 105
Asn Ser Asn Lys Lys Lys Arg Asp Asp
106 110 114
SEQ. ID. NO. 12
                                                        Met Gln Asp Pro Tyr Val Lys Glu
                                                                  24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
```

Polyclonal antibodies to CD4 receptor may be obtained using the whole molecule of human CD4 receptor of the following sequence:

```
SEQ. ID. NO. 13
Met Asn Arg Gly Val Pro Phe Arg His Leu Leu Leu Val Leu Gln
  1 5 10 15
Leu Ala Leu Leu Pro Ala Ala Thr Gln Gly Lys Lys Val Val Leu
  16 20 25 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
```

The polyclonal antibodies to CD4 receptor can be obtained using a polypeptide fragment of CD4 receptor chosen, for example, from the following amino-acid sequences:

```
SEQ. ID. NO. 14
                                                                        Gly Lys Lys Val Val Leu
                                                                          26 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ. ID. NO. 15
                                                Ile Gly Leu Gly Ile Phe Phe Cys Val
                                                412 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ. ID. NO. 16
                                                                        Gly Lys Lys Val Val Leu
                                                                                26 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
SEQ. ID. NO. 17
                                                                                                                Asp
  91 95 100 105
Thr Tyr Ile Cys Glu Val Glu Asp Gln Lys Glu Glu Val Gln
106 110 115 119
SEQ. ID. NO. 18
                                                                        Lys Glu Glu Val Gln Leu
                                                                        115 120
Leu Val Phe Gly Leu Thr Ala Asn Ser Asp Thr His Leu Leu Gln
121 125 130 135
Gly Gln Ser Leu
136 139
```

The polyclonal antibodies to CD8 receptor may also be obtained by a similar methodology to the methodology described for CD4 receptor antibodies using an adjuvant. Polyclonal antibodies to CD8 receptor may be obtained using the whole molecule of CD8 receptor of the following sequence:

```
SEQ ID NO: 19
Met Ala Leu Pro Val Thr Ala Leu Leu Leu Pro Leu Ala Leu Leu
  1 5 10 15
Leu His Ala Ala Arg Pro Ser Gln Phe Arg Val Ser Pro Leu Asp
  16 20 25 30
Arg Thr Trp Asn Leu Gly Glu Thr Val Glu Leu Lys Cys Gln Val
  31 35 40 45
Leu Leu Ser Asn Pro Thr Ser Gly Cys Ser Trp Leu Phe Gln Pro
  46 50 55 60
Arg Gly Ala Ala Ala Ser Pro Thr Phe Leu Leu Tyr Leu Ser Gln
  61 65 70 75
Asn Lys Pro Lys Ala Ala Glu Gly Leu Asp Thr Gln Arg Phe Ser
  76 80 85 90
Gly Lys Arg Leu Gly Asp Thr Phe Val Leu Thr Leu Ser Asp Phe
  91 95 100 105
Arg Arg Glu Asn Glu Gly Tyr Tyr Phe Cys Ser Ala Leu Ser Asn
106 110 115 120
Ser Ile Met Tyr Phe Ser His Phe Val Pro Val Phe Leu Pro Ala
121 125 130 135
Lys Pro Thr Thr Thr Pro Ala Pro Arg Pro Pro Thr Pro Ala Pro
136 140 145 150
Thr Ile Ala Ser Gln Pro Leu Ser Leu Arg Pro Glu Ala Cys Arg
151 155 160 165
Pro Ala Ala Gly Gly Ala Val His Thr Arg Gly Leu Asp Phe Ala
166 170 175 180
Cys Asp Ile Tyr Ile Trp Ala Pro Leu Ala Gly Thr Cys Gly Val
181 185 190 195
Leu Leu Leu Ser Leu Val Ile Thr Leu Tyr Cys Asn His Arg Asn
196 200 205 210
Arg Arg Arg Val Cys Lys Cys Pro Arg Pro Val Val Lys Ser Gly
211 215 220 225
Asp Lys Pro Ser Leu Ser Ala Arg Tyr Val
226 230 235
```

The use of CD8 receptor fragments as antigen is also contemplated. The suitable sequences for such antigen are as follow:

```
SEQ ID NO: 20
                                                                                Pro Leu Ala Leu Leu
                                                                                  11 15
Leu His Ala Ala Arg Pro Ser Gln Phe Arg Val Ser Pro Leu Asp
  16 20 25 30
SEQ ID NO: 21
                                        Ala Glu Gly Leu Asp Thr Gln Arg Phe Ser
                                          81 85 90
Gly Lys Arg Leu Gly Asp Thr Phe Val Leu
  91 95 100
SEQ ID NO: 22
Ser Ile Met Tyr Phe Ser His Phe Val Pro Val Phe Leu Pro Ala
121 125 130 135
Lys Pro Thr Thr Thr
136 140
SEQ ID NO: 23
                                        Val Ile Thr Leu Tyr Cys Asn His Arg Asn
                                        201 205 210
SEQ ID NO: 24
                                                                                Val Val Lys Ser Gly
                                                                                221 225
Asp Lys Pro Ser Leu Ser Ala Arg Tyr Val
226 230 235
```

The exemplary procedure for preparation of the starting polyclonal antibodies to IFN-gamma and/or CD4 receptor and/or CD8 receptor may be described as follows. In 7-9 days before blood sampling, 1-3 intravenous injections of the desired antigen are made to the rabbits to increase the level of polyclonal antibodies in the rabbit blood stream. Upon immunization, blood samples are taken to test the antibody level. Typically, the maximum level of immune reaction of the soluble antigen is achieved within 40 to 60 days after the first injection of the antigen. Upon completion of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in a 50 ml centrifuge tube. Product clots formed on the tube sides are removed with a wooden spatula, and a rod is placed into the clot in the tube center. The blood is then placed in a refrigerator for one night at the temperature of about 40° C. On the following day, the clot on the spatula is removed, and the remaining liquid is centrifuged for 10 min at 13,000 rotations per minute. Supernatant fluid is the target antiserum. The obtained antiserum is typically yellow. 20% of NaN₃ (weight concentration) is added in the antiserum to a final concentration of 0.02% and stored before use in frozen state at the temperature of −20° C. or without NaN₃ at the temperature of −70° C. To separate the target antibodies to IFN-gamma, CD4 receptor or CD8 receptor from the antiserum, the following solid phase absorption sequence is suitable:

10 ml of the antiserum of rabbits is diluted twofold with 0.15 M NaCl, after which 6.26 g Na₂SO₄ is added, mixed and incubated for 12-16 hours at 4° C. The sediment is removed by centrifugation, diluted in 10 ml of phosphate buffer and dialyzed against the same buffer during one night at ambient temperature. After the sediment is removed, the solution is applied to a DEAE-cellulose column balanced by phosphate buffer. The antibody fraction is determined by measuring the optical density of the eluate at 280 nm.

The isolated crude antibodies are purified using affine chromatography method by attaching the obtained antibodies to IFN-gamma, CD4 or CD8 antigen located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to IFN-gamma, CD4 or CD8 receptor is 0.5 to 5.0 mg/ml, preferably, 2.0 to 3.0 mg/ml.

The activated-potentiated form of an antibody to IFN-gamma or CD4 receptor or CD8 receptor may be prepared from an initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution) of a neutral solvent, starting with a concentration of the initial solution of antibody in the solvent, preferably, water or a water-ethyl alcohol mixture, in the range from about 0.5 to about 5.0 mg/ml, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to IFN-gamma or CD4 receptor or CD8 receptor with the concentration of 3.0 mg/ml is diluted in 99 parts of neutral aqueous or aqueous-alcohol solvent (preferably, 15%-ethyl alcohol) and then vertically shaked many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to gamma interferon with the concentration of 3.0 mg/ml in 99 parts of a neutral solvent in different containers, which is equivalent to the centesimal homeopathic dilution C12. Similar procedures with the relevant dilution factor are performed to obtain dilutions C30, C50 and C 200. The intermediate dilutions may be tested in a desired biological model to check activity. The preferred activated-potentiated forms for the composition of the invention are a mixture of C12, C30, and C50 dilutions or C12, C30 and C200 dilutions. When using the mixture of various homeopathic dilutions (primarily centesimal) of the active substance as biologically active liquid component, each component of the composition (e.g., C12, C30, C50, C200) is prepared separately according to the above-described procedure until the next-to-last dilution is obtained (e.g., until C11, C29, and C199 respectively), and then one part of each component is added in one container according to the mixture composition and mixed with the required quantity of the solvent (e.g. with 97 parts for centesimal dilution).

It is possible to use the active substance as mixture of various homeopathic dilutions, e.g. decimal and/or centesimal (D20, C30, C100 or C12, C30, C50 or C12, C30, C200, etc.), the efficiency of which is determined experimentally by testing the dilution in a suitable biological model, for example, in models described in the examples herein.

In the course of potentiation and concentration decrease, the vertical shaking may be substituted for external exposure to ultrasound, electromagnetic field or any similar external impact procedure accepted in the homeopathic art.

For preparation of, for example, the (potentiated) antibodies to the human IFN-γ or CD4 or CD8 in a liquid pharmaceutical form in a very-low-dose the antiretroviral agent is entered in an effective therapeutic dose based, for, instance, on AZT. The obtaining of the aforementioned pharmaceutical composition in a solid form may be accomplished by the saturation of the solid antiviral ingredient (inclusive of neutral carrier) at the stage of its preparation with the solution of the activated (potentiated) antibodies to the protein or peptide of the immune system, which interact with HIV or content and/or fictional activity of which is changed due to the HIV infection (for example the granules of the neutral carrier (predominantly lactose) is saturated with the solution of the activated (potentiated) antibodies, which jointly with the active substance and additive agents generate pressure necessary for creation of a solid form preparation.

The combined use by means of simultaneous introduction of the components of the composition in the form of two separate preparations (one or several antiviral medications or active substances in an effective therapeutic dose and the activated (potentiated) antibodies to the protein or peptide of the immune system) is also possible.

Preferably, the pharmaceutical composition of the invention may be in the form of a liquid or in the solid unit dosage form. The preferred liquid carrier is water or water-ethyl alcohol mixture. Most preferably, the pharmaceutical composition of the invention is in the form of a single solid oral dosage form containing both active ingredients.

The solid unit dosage form of the pharmaceutical composition of the invention may be prepared by impregnating a solid, pharmaceutically acceptable carrier with the mixture of the activated potentiated form aqueous or aqueous-alcohol solutions of active component. Alternatively, the carrier may be impregnated consecutively with each requisite dilution. Both orders of impregnation are acceptable. The antiretroviral compound, preferably AZT, may be introduced in the preparation process before, during or after the impregnation of the solid carrier.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies to IFN-gamma or CD4 receptor or CD8 receptor. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono- olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose, magnesium stearate and citric acid.

With respect to the introduction of the activated-potentiated component of the pharmaceutical composition of the invention, an exemplary methodology may be as follows. 100-300 μm granules of lactose are impregnated with aqueous or aqueous-alcoholic solutions of the activated-potentiated form of antibodies to IFN-gamma or CD4 receptor or CD8 receptor in the ratio of 1 kg of antibody solution to 5 or 10 kg of lactose (1:5 to 1:10). To effect impregnation, the lactose granules are exposed to saturation irrigation in the fluidized boiling bed in a boiling bed plant (e.g. “Hüttlin Pilotlab” by Hüttlin GmbH) with subsequent drying via heated air flow at a temperature below 40° C. The estimated quantity of the dried granules (10 to 34 weight parts) saturated with the activated potentiated form of antibodies is placed in the mixer, and mixed with 25 to 45 weight parts of “non-saturated” pure lactose (used for the purposes of cost reduction and simplification and acceleration of the technological process without decreasing the treatment efficiency), together with 0.1 to 1 weight parts of magnesium stearate, and 3 to 10 weight parts of microcrystalline cellulose. The obtained tablet mass is uniformly mixed, and tableted by direct dry pressing (e.g., in a Korsch—XL 400 tablet press) to form 150 to 500 mg round pills, preferably, 300 mg. After tableting, 300 mg pills are obtained that are saturated with aqueous-alcohol solution (3.0-6.0 mg/pill) of the activated-potentiated form of antibodies to IFN-gamma or CD4 receptor or CD8 receptor in the form of a mixture of centesimal homeopathic dilutions C12, C30, and C50 or a mixture of centesimal homeopathic dilutions C12, C30 and C200.

Any suitable route of administration can be employed for providing the patient with a therapeutically or prophylactically effective dose of an active ingredient. For example, oral, mucosal (e.g., nasal, sublingual, buccal, rectal, vaginal), parenteral (e.g., intravenous, intramuscular), transdermal, and subcutaneous routes can be employed. The preferred route of administration is oral.

The dosage forms of the invention include, but are not limited to, tablets, caplets, troches, lozenges, dispersions, suspensions, suppositories, ointments, cataplasms (poultices), pastes, powders, dressings, creams, plasters, solutions, capsules, soft elastic gelatin capsules, and patches. In practical use, the active ingredient may be, for example, combined in an intimate admixture with a pharmaceutical carrier(s) according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, preferably without employing the use of lactose. For example, suitable carriers include powders, capsules, and tablets, with the solid oral preparations being preferred over the liquid preparations. Because of their ease of administration, tablets and capsules represent the most advantageous oral dosage unit forms, in which case solid pharmaceutical carriers are employed. If desired, tablets can be coated by standard aqueous or nonaqueous techniques.

In addition to the common dosage forms set out above, an active ingredient can also be administered by controlled release means or delivery devices that are well known to those of ordinary skill in the art, such as those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, the disclosures of which are incorporated herein by reference. These dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. The invention thus encompasses single unit dosage forms suitable for oral administration such as, but not limited to, tablets, capsules, gelcaps, and caplets that are adapted for controlled-release.

Pharmaceutical compositions of the invention suitable for oral administration can be presented as discrete dosage forms, such as capsules, cachets, or tablets, or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses both lactose-containing and lactose-free pharmaceutical compositions and dosage forms. Lactose is typically used as a solid carrier in formulations of activated-potentiated forms of antibodies. See, e.g., U.S. application Ser. No. 13/135,899, PCT/RU2011/000523, U.S. Pat. No. 7,572,441, and U.S. Pat. No. 8,066,992, which are hereby incorporated by reference for the purposes stated and in their entirety. The compositions of the invention may contain little, if any, lactose or other mono- or di-saccharides. As used herein, the term “lactose-free” means that the amount of lactose present, if any, is insufficient to substantially increase the degradation rate of an active ingredient. Lactose-free compositions may comprise excipients which are well known in the art and are listed in the USP (XXI)/NF (XVI), which is incorporated herein by reference. In general, lactose-free compositions comprise an active ingredient, a binder/filler, and a lubricant in pharmaceutically compatible and pharmaceutically acceptable amounts. The pharmaceutical compositions may comprise the active ingredients, microcrystalline cellulose, pre-gelatinized starch, and magnesium stearate.

The pharmaceutical compositions of the invention may include binders. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, for example, the materials sold as AVICEL-PH-101, AVICEL-PH-1103 AVICEL RC-581, and AVICEL-PH-105 (available from FMC Corporation, American Viscose Division, Avicel Sales, Marcus Hook, Pa., U.S.A.). An exemplary suitable binder is a mixture of microcrystalline cellulose and sodium carboxymethyl cellulose sold as AVICEL RC-581. Suitable anhydrous or low moisture excipients or additives include AVICEL-PH-103 and Starch 1500 LM.

The pharmaceutical compositions of the invention may include fillers. Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder/filler in pharmaceutical compositions of the present invention is typically present in about 50 to about 99 weight percent of the pharmaceutical composition.

The pharmaceutical compositions of the invention may include disintegrants. Disintegrants are used in the compositions of the invention to provide tablets that disintegrate when exposed to an aqueous environment. Too much of a disintegrant will produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form the dosage forms of the compounds disclosed herein. The amount of disintegrant used varies based upon the type of formulation and mode of administration, and is readily discernible to those of ordinary skill in the art. Typically, about 0.5 to about 15 weight percent of disintegrant, preferably about 1 to about 5 weight percent of disintegrant, can be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

The pharmaceutical compositions of the invention may include lubricants. Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), or mixtures thereof. A lubricant can optionally be added, typically in an amount of less than about 1 weight percent of the pharmaceutical composition.

The invention is further illustrated with reference to the appended non-limiting examples.

### EXAMPLES

### Example 1

The assessment of antiretroviral activity of ultra low-dose of rabbit polyclonal antibodies to CD4 receptor (a mixture of homoeopathic dilutions C12+C30+C50) (ULD Ab CD4)), was carried out using human peripheral blood mononuclear cells infected with the strain HIV-1 LAI in vitro.

Human peripheral blood mononuclear cells were isolated from blood of a seronegative healthy donor by centrifugation on a Ficoll-Hypaque density gradient. The cells were stimulated for 3 days with 1 μg/mL of phytohemagglutinin P and 5 IU/mL of recombinant human interleukin-2.

In order to assess antiretroviral activity the products were placed in a well containing 100 μL of activated mononuclears 24 hours before or 15 min after cell infection with the strain HIV-1-LAI at the dose of 100 TCID50 (50 μL inoculum of the strain HIV-1-LAI). Before adding to a well, ULD Ab CD4 (12.5 μL) or reference azidotimidine (1000 nM) were mixed with RPMI1640 medium (DIFCO) to achieve a final probe volume of 50 μL

The supernatant fluids were collected on day 7 after infection of cells. The products' activity was measured by the inhibition of HIV replication which was assessed by the level of core nucleocapsid protein p24 in the supernatant fluid from human peripheral blood mononuclear cells using Retrotek Elisa kit.

It was shown that ULD Ab CD4 inhibited HIV replication by 86±10% when added to a well 24 hours before the infection, a and by 51±3% when added to a well 15 min after the infection of cells with the strain HIV-1 LAI. Azidotimidine at a dose of 1000 nM inhibited HIV replication by 99±0 and 99±1% added to a well 24 hours before and 15 min after the infection of cells with the strain HIV-1 LAI, respectively.

Thus, this experimental model demonstrated the antiretroviral activity of ultra low-doses of rabbit polyclonal antibodies to CD4 (a mixture of homoeopathic dilutions C12+C30+C50.

### Example 2

The assessment of antiretroviral activity of ultra low-dose rabbit polyclonal antibodies to CD4 (a mixture of homoeopathic dilutions C12+C30+C50) (hereinafter referred to as “ultra low-dose antibodies to CD4) was carried out using human peripheral blood mononuclear cells infected with the strain HIV-1 LAI in vitro. Azidothymidine (Sigma—AZ169-100 mg, lot 107K1578) was used as a comparator product.

Human peripheral blood mononuclear cells were isolated from blood of a seronegative healthy donor by centrifugation on a Ficoll-Hypaque density gradient. The cells were stimulated for 3 days with 1 μg/mL of phytohemagglutinin P and 5 IU/mL of recombinant human interleukin-2 in RPMI1640 (DIFCO) medium supplemented with 10% fetal calf serum (the complement was removed by heating for 45 minutes at 56° C.), 1% antibiotic solution (PSN Gibco containing 50 μg/mL of penicillin, 50 μg/mL of streptomycin and 100 μg/mL of neomycin).

In order to assess antiretroviral activity the products were placed in a well 15-30 minutes after cells infection with the strain HIV-1-LAI at the dose of 100 TCID50 (50 μL inoculum of the strain HIV-1-LAI). Supernatant fluids used to assess the effect of products on the inhibition of HIV replication were also collected on day 7 after cells infection.

Before placing in a well, which contained 150 μL of cell culture, ultra low-dose antibodies to CD8 were diluted with RPMI1640 (DIFCO) medium at a 4-fold dilution (at a 1/4 dilution) to a final volume of 50 μL. Azidothymidine was diluted with RPMI1640 (DIFCO) medium to yield a 8 nM concentration.

The products' efficiency was established by the inhibition of HIV replication which was assessed by HIV-reverse transcriptase activity in the supernatant fluid from human peripheral blood mononuclear cells using the HIV RT RetroSys kit made by INNOVAGEN (Lot 10-059C). The supernatant fluid of cells, to which test products or azidothymidine were not inoculated, was used as control to calculate the percentage of inhibition of HIV replication (see Table 1).

TABLE 1
Antiretroviral activity of ultra low-dose antibodies
to CD4 using human peripheral blood mononuclear cells
infected with the strain HIV-1-LAI in vitro

| | | Inhibition of HIV-reverse |
|---|---|---|
| | Medium Dilution | transcriptase activity |
| | Ratio RPMI1640 | (% of control) |
| Product | (DIFCO) | Day 7 |
| Ultra-low dose of | ¼ | 78 ± 6 |
| antibodies to CD4 | | |
| receptor | | |
| Azidothymidine | — | 58 ± 7 |
| (8 nM) | | |

Thus, this experimental model demonstrated the antiretroviral activity of ultra low-dose rabbit polyclonal antibodies to CD4 receptor (a mixture of homoeopathic dilutions C12+C30+C50).

### Example 3

The experimental study involved the affine purified rabbit polyclonal antibodies to the human IFN-gamma. The affine purified rabbit polyclonal antibodies to the human gamma interferon were used for development of the (potentiated) antibodies to the gamma interferon in a very-low-dose in the form of serial dilutions C12+C30+C50, obtained in accordance with a homeopathic technology (hereinafter referred to as Ab to IFN-γ in VLD).

Antiviral activity of the composition under review Ab to IFN-γ in VLD and azidothymidine (preparation, based on an active substance of Zidovudine) was studied within the context of mononuclear cells of the human blood, infected in vitro with the HIV-strain-1-LAI. The efficiency of replicating inhibition of HIV was assessed according to the content of a main nucleocapsid protein p24 HIV in the supernatant fluid of the mononuclear cells of the human peripheral blood.

Mononuclear leucocytes of the human peripheral blood were extricated from the blood of the antibody-negative donors with the use of centrifuging in the Ficoll-Paque density gradient. The cells were activated within 3 days with the use of 1 mkg/ml of phytohemagglutinin P and ME/ml of the Interleukin-2 human recombinant.

In order to assess the antiretroviral activity, the wells, containing 100 mcL of activated mononuclear leucocytes of the human peripheral blood, 24 prior to or 15 minutes after the contamination of cells with HIV-strain-1-LAI in the dose of 100 TCID50 (50 mcL of the HIV-strain-1-LAI inoculum), were introduced the combination of Ab to IFN-γ in VLD with azidothymidine (azidothymidine dose in combination 10 nM) (12. mcL) or with azidothymidine in the dose of 1000 nM (active comparator), mixed with environment RPM11640 (DIFCO) prior to the gaining of the final volume of 50 mcL.

Supernatant fluid of the cell culture was collected on the seventh (7) day subsequent to the contamination. The efficiency of the preparations was assessed according to the inhibition of HIV reaction, in its turn assessed in accordance with the content of a main nucleocapsid protein p24 HIV in the supernatant cells by method of IFA, immunofluorescent assay (Retrotek Elisa kit).

It was found, that azidothymidine in the dose of 1000 nM inhibited the HIV reaction by 99±0 and 99±1% by the introduction into the wells 24 prior to and 15 minutes after the contamination of cells with HIV-strain-1-LAI, respectively. The combination of Ab to IFN-γ in VLD with azidothymidine in the dose of 10 nM, which is 100 lower than the dose of azidothymidine by monotherapy, inhibited the HIV reaction by 80±9% by the introduction into the wells 15 minutes after the contamination of cells with HIV-strain-1-LAI, respectively.

Consequently, the in vitro study showed, that combined use of azidothymidine with Ab to IFN-γ in VLD allows reducing the azidothymidine dose by 100 times and preserving its initial high antiviral efficiency.

Note: TCID50—dose, infecting 50% of the tissue culture cells.

### Example 4

Materials And Methods

Clinical and laboratory study were carried out within the Volgograd State Medical University on the basis of State health care institution “Volgograd Regional Centre on HIV/AIDS and Infectious Diseases Prevention” (head physician—Gorshkova N.V.) within the period 2007-2009.

Characteristics of the Patients Groups, Terms of the Research.

The research was carried out within the framework of a randomized controlled prospective study in parallel groups. It included the patients, who were willing to sign an informed consent form. The clinical research protocol was approved by an Independent Ethics Committee. All the participants were familiarized to the aims, tasks and nature of the present research and all the relevant information on “Anaferon”. The participation in the research was voluntary. The group involved in the research comprised 83 patients (age ranging from 18 to 47, 37% of women) diagnosed with HIV infection. The control group involved 20 healthy volunteers (age ranging from 20 to 22, 70% of women, 30% of men) with no traces of chronic diseases, traumas, surgical interventions and experiencing no acute conditions within the previous 6 months. Randomization of patients was carried out according to the random number table.

Criteria of Patients' Involvement into the Research.

Patients included to the groups under investigation were involved into the research according to the criteria as follows:

1. Age ranging from 18 to 55;

2. Patients diagnosed with HIV infection;

3. Supervision by the State health care institution “Volgograd Regional Centre on HIV/AIDS and Infectious Diseases Prevention”

Criteria of Patients' Exclusion from the Research.

The patients suffering from hepatic cirrhosis, viral hepatitis type C, active stage serious concomitant diseases, pregnant women and people who use intravenous (IV) drugs were not involved into the research. Patients that did not comply with the recommendations on regular use of the preparation and timely examinations, as well as unwillingness to continue the treatment course were excluded from the research. Participation in other researches was also a basis for exclusion.

The patients were subdivided into five (5) groups:
Group I—HIV infected patients, treated with “Anaferon” without ARVT (11 patients)
Group II—HIV infected patients, treated with “Anaferon” and ARVT (16 patients)
Group III—HIV infected patients, not treated with “Anaferon” and ARVT (18 patients)
Group IV—HIV infected patients, treated with ARVT without “Anaferon” (22 patients)
Group V—HIV infected patients at the 4B stage of HIV infection, treated with ARVT and “Anaferon” (14 patients)

The terms of treatment are chosen by reference to seasonal aggravation of the underlying viral diseases of HIV infected patients.

The patients started using “Anaferon” prior to such aggravations according to two schemes: patients of the Group I and Group II used “Anaferon” in the phylactic dosage of one (1) tablet per day within the period of one and a half (1.5) months. Patients of the Group V used “Anaferon” in the curative dosage of one tablet every thirty (30) minutes within first two (2) hours (5 tablets in total), than three (3) more tablets taken at equal time intervals within the first 24 hours and than (1) tablet three times a day within the period of one and a half (1.5) months.

Assessment of the General Condition of HIV Infected Patients Prior and Subsequent to the Treatment with “Anaferon”.

In order to assess the influence of “Anaferon” on the life quality of the patients, included to the aforementioned groups, they were offered to independently fill in a questionnaire, comprising 14 questions, regarding the somatic and psychic status of the volunteers, which was drafted ob the basis of the international questionnaire on general health SF-36 and adjusted to the research under review. Results of the questioning were analyzed in accordance with the attached methodology.

Blood Sampling.

The samples of blood were taken in the morning under fasting conditions prior to the medications intake and curative procedures into the vacuum tubes made of ethylenediaminotetraacetic acid (EDTA)—Green Vac Tube (Green Cross, Korea).

Lymphocite immunophenotyping. Phenotyping of circulating lymphocytes of the peripheral blood was carried out on the ductal cytofluorometer FACSCount (Becton Dickinson, the USA) with the use of conventional test-systems FACSCount Reagent Kit, comprising CD3, CD4, CD8 antibodies marked with FITC, PE fluorochromes.

Assessment of Interleukin-2 (IL-2), Interleukin-4 (IL-4), IFN-γQuantity.

Quantitative determination of cytotoxicants within the studied samples was carried out with the use of ELISA technique and test-system (“Biosource”, Invitrogen, the USA) in accordance with the attached protocols.

Assessment of the Viral Load.

The quantitative analyses of the type I immune deficiency virus involved COBAS AMPLICOR HIV-1 MONITOR Kit, version 1.5 for automatic PCR-analyzer COBAS AMPLICOR (Roche, Switzerland). The kit is used as a test on nucleic acids amplification in vitro in order to quantitatively identify the type I immune deficiency virus and its ribonucleic acid (RNA) forms within the clinical samples. The test is based on the polymerase chain reaction used for nucleic acids amplification, as well as on nucleic acids hybridization used for quantitative determination of RNA HIV-1 in the plasma, extricated from the whole blood.

Statistical processing of the research results was carried out with the use of Microsoft Excel 2003, Statsoft Statistica 6.0, SPSS Statistics 17.0.

Data, subject to normal distribution law, was used with a view to mean and standard error of mean. Data, not subject to normal distribution law, was described by means of theoretical median (Me), as well as the first and the third quartile (Q1, Q3). Critical significance level (p) within the hypothesis verification equaled 0.05. The comparison of groups for Gaussian sample involved the Student's t-test, while nonparametric sampling required the use of Mann-Whitney U-test and Wilkockson t-test. The normalcy of distribution assessment was carried out on the basis of the Shapiro-Wilk W-test together with the visual evaluation of distribution bar chart, gained with the use of Statsoft Statistica 6.0 software.

Results

No adverse drug reactions were traced in patients, treated with “Anaferon”.

Assessment of Circulating Lymphocytes Subpopulations.

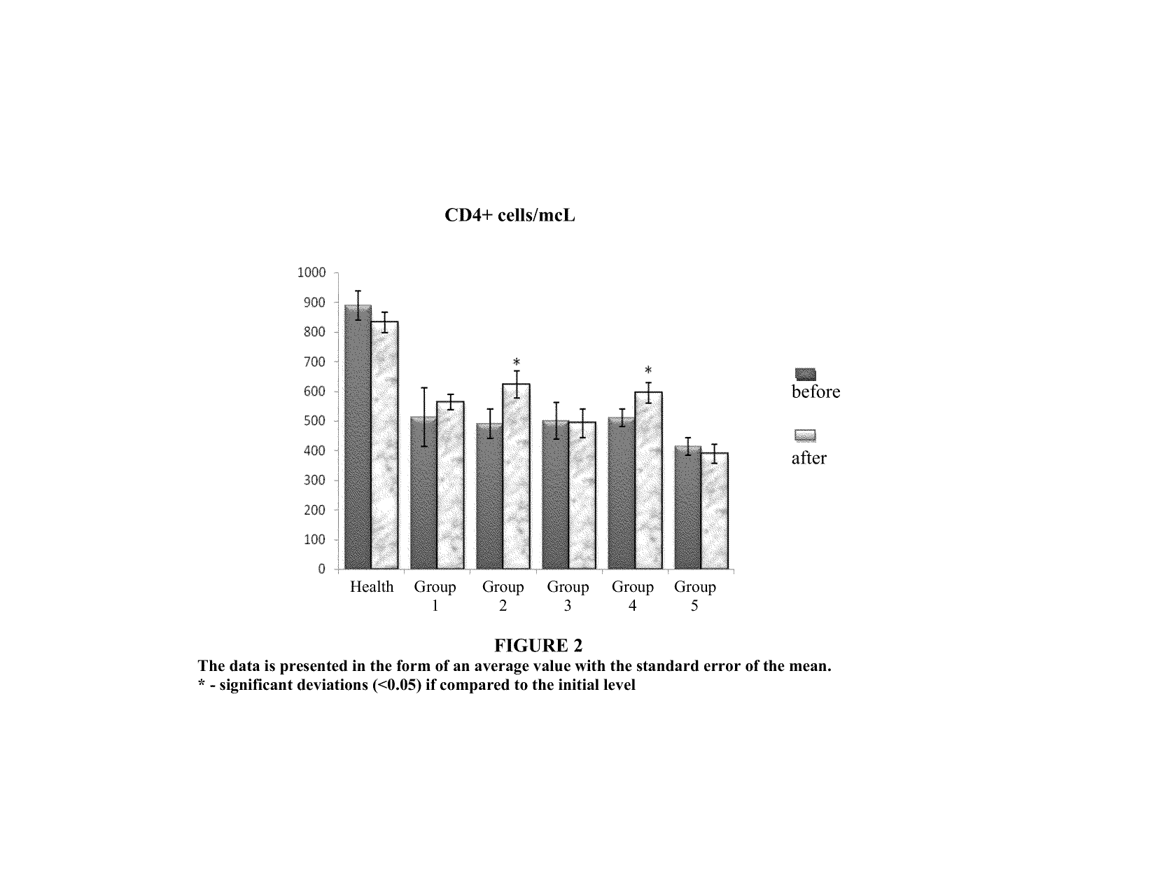

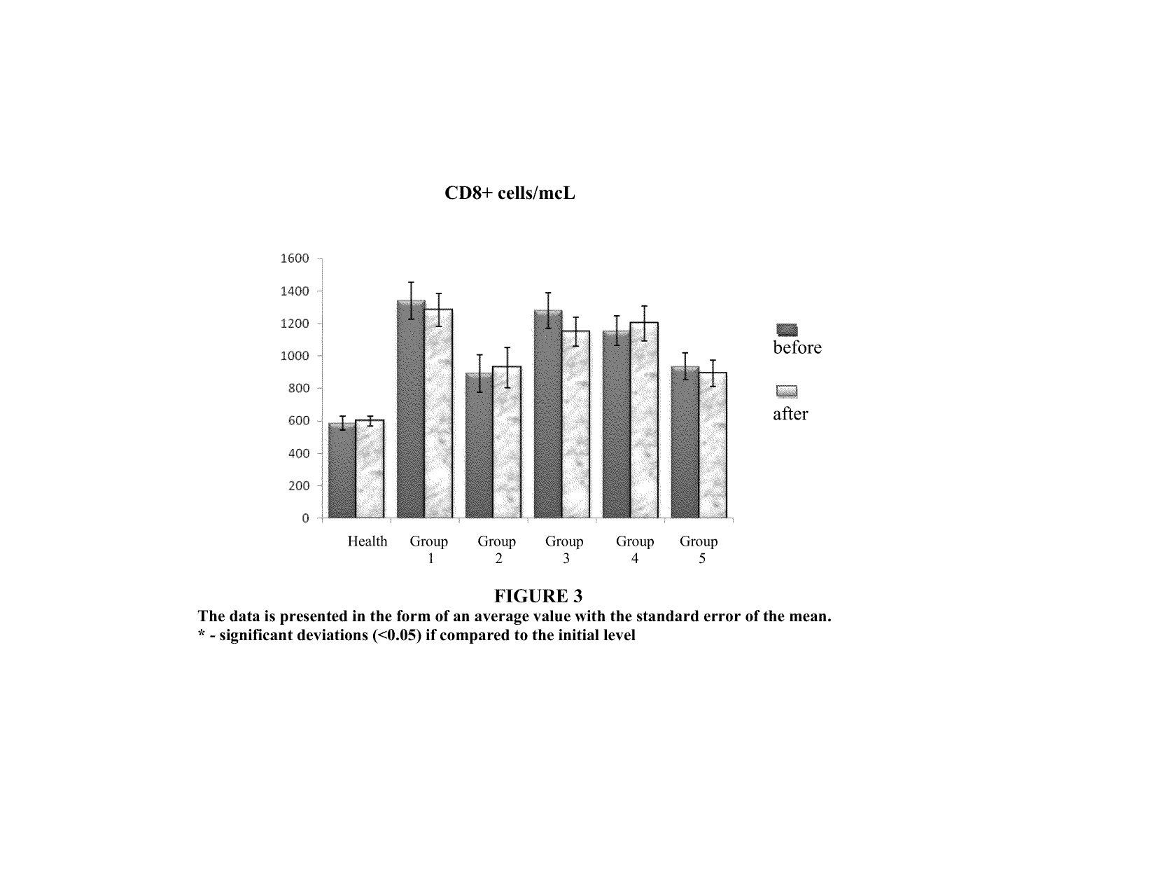

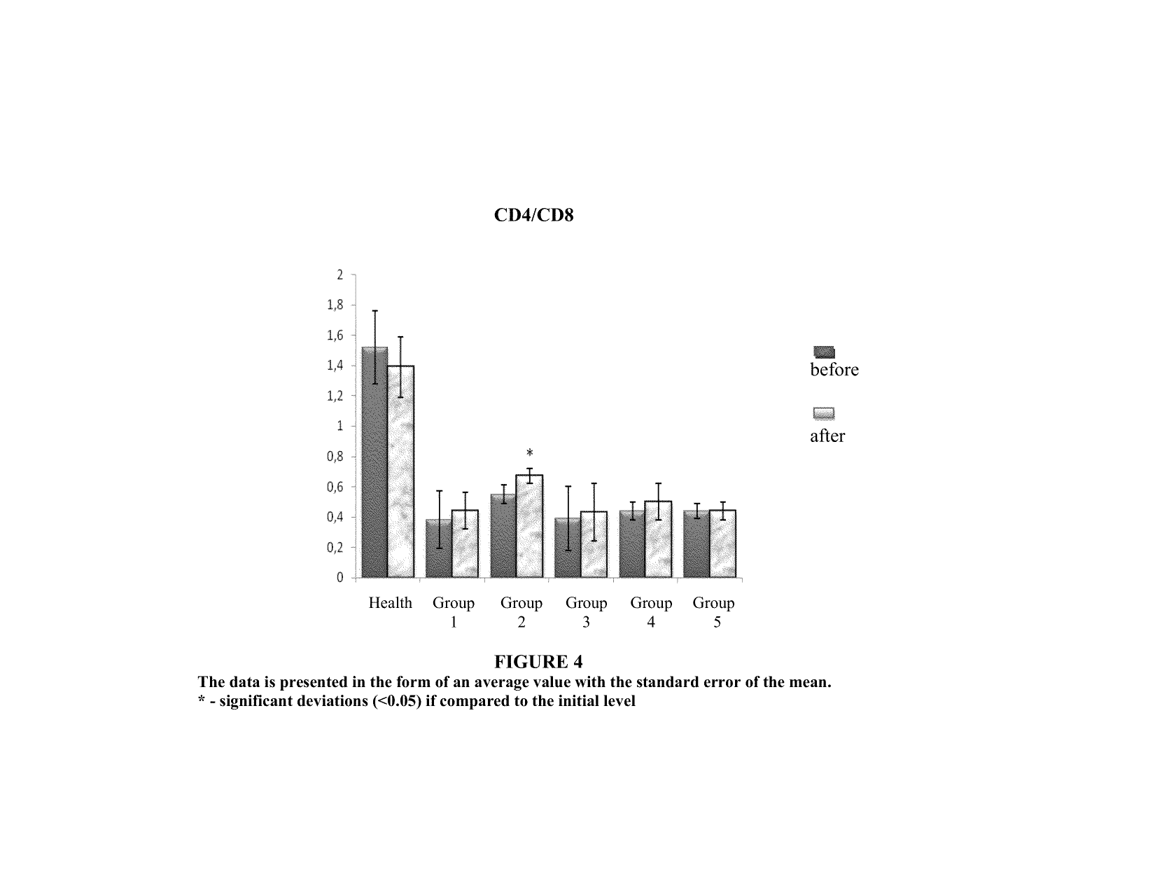

Immune status study in patients included to the groups under review allowed revealing significant differences if compared to the health group. Prior to the use of the preparation amount of CD4 cells was significantly reduced in all the patients (group No. 1: 513±31 cells/mcL; group No. 2: 511±50 cells/mcL; group No. 3: 500±62 cells/mcL; group No. 4: 510±29 cells/mcL; group No. 5: 414±29 cells/mcL to compare with 849±49 cells/mcL in the control group, p<0.05) (FIG. 2). On the background of reduction in T-helpers level the significantly high content (p<0.05) of CD8 lymphocytes in the peripheral blood of patients included to all the groups was observed (group No. 1: 1340±114 cells/mcL; group No. 2: 893±116 cells/mcL; group No. 3: 1280±109 cells/mcL; group No. 4: 1155±92 cells/mcL; group No. 5: 936±83 cells/mcL to compare with 586±43 cells/mcL in the control group, p<0.05) (FIG. 3). The study of CD8 lymphocytes level allowed revealing significant reduction (p<0.05) in immunoregulatory index CD4/CD8 (group No. 1: 0.78±0.19; group No. 2: 0.65±0.06; group No. 3: 0.41±0.21; group No. 4: 0.48±0.06; group No. 5: 0.48±0.05 to compare with 1.59±0.14 in the health group) (FIG. 4). In such a way, the difference between HIV infected patients and volunteers from the health group in terms of CD3 lymphocytes amount may be explained with a view to CD8 cells increase on the background of a proportional reduction in CD4 cells, what constitutes a natural process of the immune system suppression on the background of HIV infection.

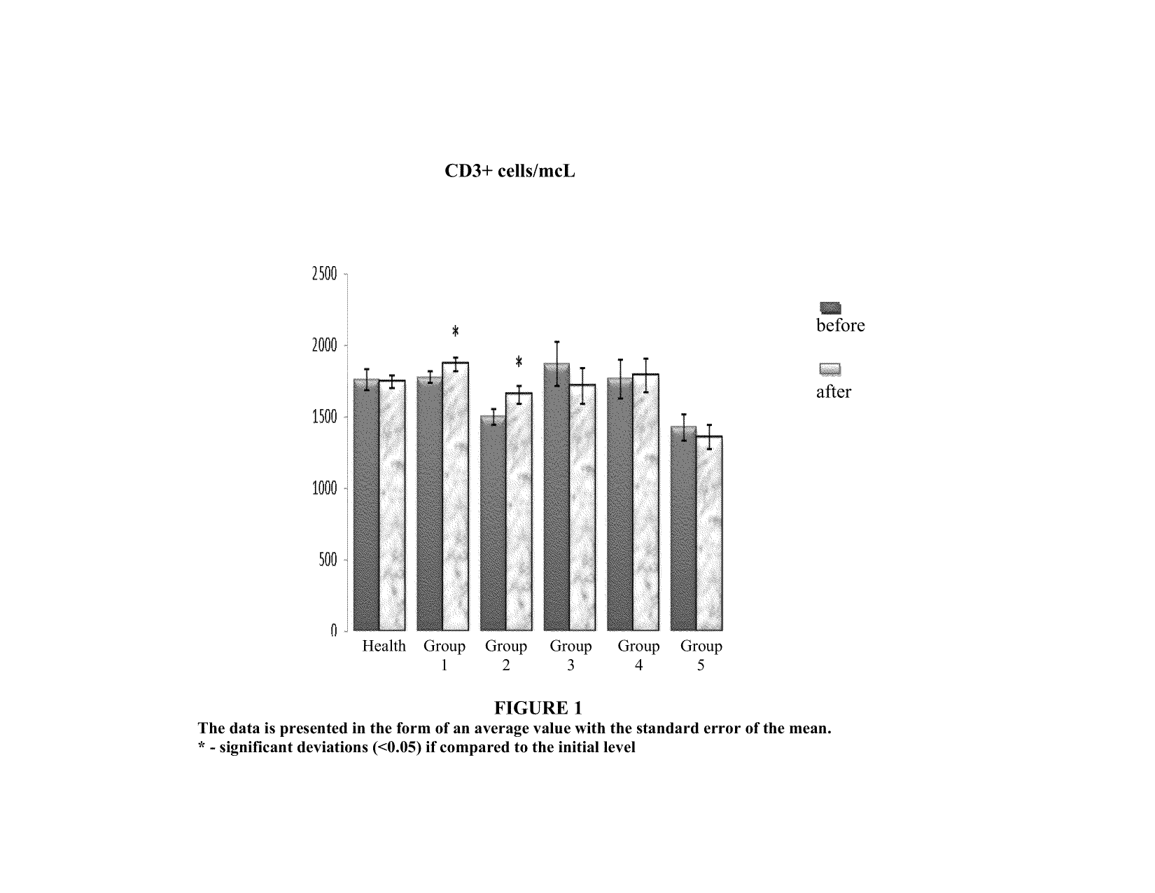

Research of the cellular components of immune system in HIV infected patients upon the cessation of “Anaferon” use allowed identifying the difference between CD4 lymphocytes amount prior to the use of preparation in groups No. 2 (623±45 and 595±35 respectively), as well as significant deviations (p<0.05) from the health groups (FIG. 2). Amount of CD8 lymphocytes also was not a subject to change upon the use of the dug; amount of sells also revealed a significant increase (p<0.05) in all the groups (group No. 1: 1284±103 cells/mcL; group No. 2: 929±123 cells/mcL; group No. 5: 893±82 cells/mcL to compare with 599±30 cells/mcL in the control group) (FIG. 3). General amount of CD3 cells upon the cessation of the course was increased in group No. 1 (1868±48 cells/mcL) and group No. 2 (1654±63 cells/mcL); significant deviations if compared to the health groups were revealed in group No. 5, where the amount of T-lymphocytes was drastically reduces, as prior to the use of the preparation (FIG. 1). Immunoregulatory index CD4/CD8 changed subsequent to the use of “Anaferon” in patients included to group No. 2 where it totaled 0.67±0.05. The rest of the groups revealed no changes in the dynamics of CD4/CD8 index, it remained significantly low (p<0.05) if compared to the control group (group No. 1: 0.44±0.12; group No. 3: 0.43±0.19; group No. 4: 0.50±0.12; group No. 5: 0.49±0.06 to compare with 1.59±0.14 in the health group) (FIG. 4).

In such a way, the influence of “Anaferon” on the quantitative representation of T-lymphocytes in HIV infected patients is traced due to an increase in CD4 cells amount provided the combined use with the antiviral medication. Having considered a reduction in viral load in patients, treated with “Anaferon” in combination with ARVT, it shall be emphasized, that an increase in T-helpers amount is attributed to the reinforcement of such cells population at the expense of healthy (uninfected) cells.

TABLE 2
Cellular components of immune system in HIV infected
patients within the dynamics of “Anaferon” use (M ± m)

| | | | |
|---|---|---|---|
| CD4+ | CD8+ | CD3+ | |
| cells/mcL | cells/mcL | cells/mcL | CD4/CD8 |

Health

| | | | | |
|---|---|---|---|---|
| before | 889 ± 49 | 586 ± 43 | 1759 ± 76 | 1.52 ± 0.24 |
| after | 832 ± 35 | 559 ± 30 | 1745 ± 42 | 1.39 ± 0.20 |

Group No. 1

| | | | | |
|---|---|---|---|---|
| before | 513 ± 98 | 1340 ± 114 | 1776 ± 41 | 0.38 ± 0.19 |
| after | 563 ± 26 | 1284 ± 103 | 1868 ± 48* | 0.44 ± 0.12 |

Group No. 2

| | | | | |
|---|---|---|---|---|
| before | 491 ± 49 | 893 ± 116 | 1498 ± 54 | 0.55 ± 0.06 |
| after | 623 ± 45* | 929 ± 123 | 1654 ± 63* | 0.67 ± 0.05* |

Group No. 3

| | | | | |
|---|---|---|---|---|
| before | 500 ± 62 | 1280 ± 109 | 1869 ± 156 | 0.39 ± 0.21 |
| after | 492 ± 49 | 1149 ± 89 | 1713 ± 127 | 0.43 ± 0.19 |

Group No. 4

| | | | | |
|---|---|---|---|---|
| before | 510 ± 29 | 1155 ± 92 | 1763 ± 137 | 0.44 ± 0.06 |
| after | 595 ± 35* | 1201 ± 107 | 1788 ± 119 | 0.50 ± 0.12 |

Group No. 5

| | | | | |
|---|---|---|---|---|
| before | 414 ± 29 | 936 ± 83 | 1426 ± 91 | 0.44 ± 0.05 |
| after | 390 ± 32 | 893 ± 82 | 1357 ± 85 | 0.44 ± 0.06 |

*significant deviations (<0.05) if compared to the initial level

Assessment of the HIV Viral Load in Patients Included to the Groups Under Investigation.

The study of immunodeficiency viral load level in the blood serum of HIV infected patients within the use of “Anaferon” revealed the reduction in quantity of RNA HIV copies in group No. 1 in 33% of the patients (average reduction in viral load totaled 9.5%). Group No. 2 showed 50%, while in group No. 5 the percentage of such patients amounted to 20%, in group No. 3-10%, and in group No. 4-31.8%. Medians of the viral load within the groups under research are illustrated in Table 3. The obtained data shows a more significant viral load reduction in group 1 (patients, treated with “Anaferon”) if compared to group 3 (patients, not treated with “Anaferon” and ARVT). Patients treated with ARVT and “Anaferon” (group 2) revealed a more expressed reduction in viral load (14.2%) as compared to patients, treated with ARVT without “Anaferon” (13.3%, group 4) (Table 3)

TABLE 3
Alterations in viral load within the researched groups of patients

| | |
|---|---|
| | Average reduction |
| Viral load copies/ml | in viral load, % |

Group No. 1 (Me [Q1-Q3])

| | | |
|---|---|---|
| Prior to the treatment course | 5813 [150-33356] | 9.5 |
| Subsequent to the treatment | 5786 [150-38359] | |
| course | | |

Group No. 2 (Me [Q1-Q3])

| | | |
|---|---|---|
| Prior to the treatment course | 4680 [274-9838] | 14.2 |
| Subsequent to the treatment | 4652 [272-8874] | |
| course | | |

Group No. 3 (Me [Q1-Q3])

| | | |
|---|---|---|
| Prior to the treatment course | 4557 [338-69497] | 5.6 |
| Subsequent to the treatment | 4575 [337-67642] | |
| course | | |

Group No. 4 (Me [Q1-Q3])

| | | |
|---|---|---|
| Prior to the treatment course | 5547 [385-58996] | 13.3 |
| Subsequent to the treatment | 5308 [338-57709] | |
| course | | |

Group No. 5 (Me [Q1-Q3])

| | | |
|---|---|---|
| Prior to the treatment course | 400 [150-1569] | −7.94 |
| Subsequent to the treatment | 400 [150-1505] | |
| course | | |

Assessment of the Cytotoxicants Status.

As it was previously known, IL-2 plays a leading role in the cellular immune system regulation. It is produced by the activated CD4 T-lymphocytes and allows enhancement of the body protection against infectious diseases by means of triggering the cells active in respect of microorganisms and viruses. A significant increase in IL-2 contents was traced in all the patients of the groups under investigation (group No. 1: 8.37 [6.61-10.13] cells/mcL; group No. 2: 5.95 [3.73-9.45] cells/mcL; group No. 3: 10.32 [7.78-13.64] cells/mcL; group No. 4: 12.11 [11.45-15.36] cells/mcL; group No. 5: 6.13 [3.02-7.82] cells/mcL to compare with 3.72[4.02-5.13] cells/mcL in the control group, p<0.05). Within the background of such an interleukin-2 increase the IL-4 contents research revealed a significant (p<0.05) cytotoxicants level reduction in the patients included to groups four and five (group 4: 0.57 [0.05-1.31] cells/mcL; group 5: 0.69 [0.11-1.27] cells/mcL to compare with 1.29[0.89-1.77] cells/mcL in the health group). IFN-γ contents in HIV infected patients was not characterized by an accurate deviation from health group, with the exception of group 5, where the serum IFN-γ concentration was drastically reduced (Table 4).

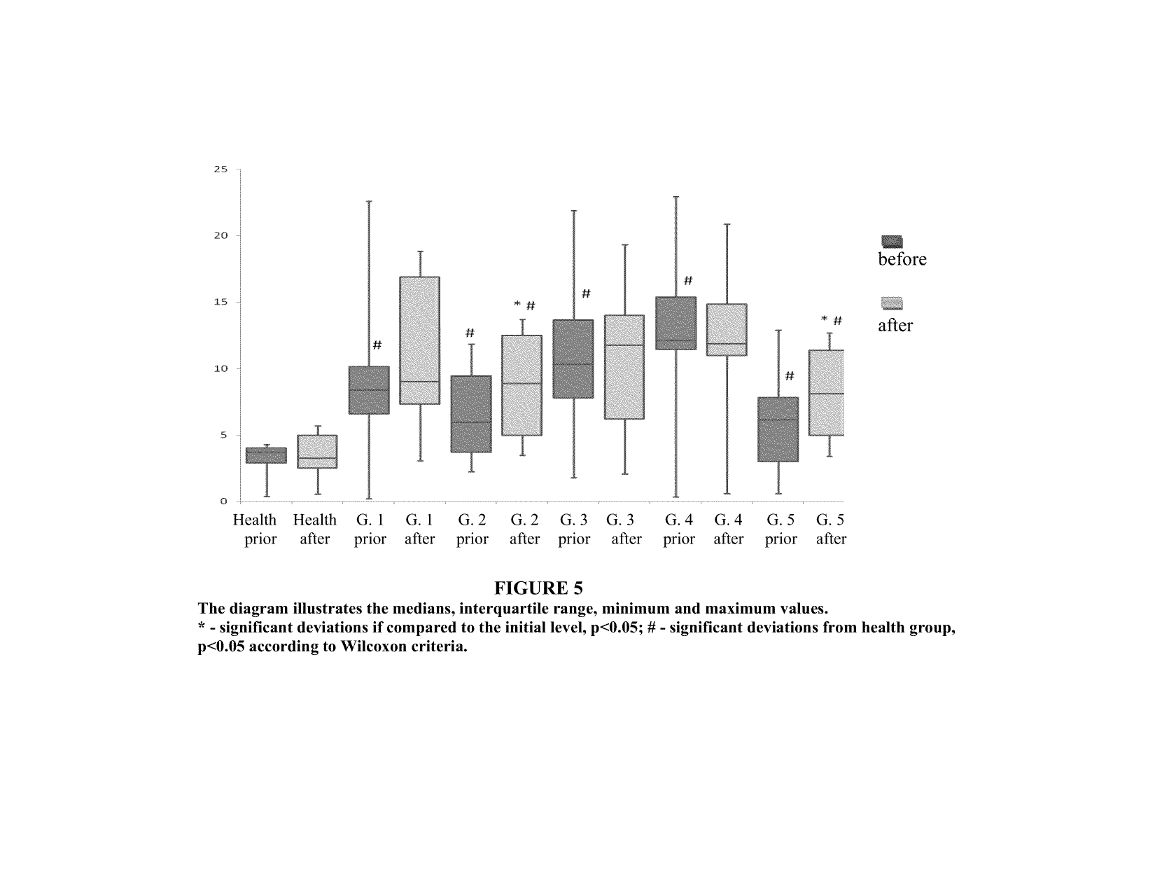

The research of interleukin-2 level within the dynamics of “Anaferon” use revealed a significant increase (p<0.05) increase in IL-2 content in patients included to groups 2 and 5 upon the cessation of the therapy course (group 2: 8.87[4.97-12.49] cells/mcL subsequent to the course compared to 2: 5.95 [3.73-9.45] cells/mcL prior to the course; group 5: 8.11 [4.98-11.39] cells/mcL subsequent to the course compared to 2: 6.13 [3.02-7.82] cells/mcL prior to the course) (FIG. 5). In such a way, the above-mentioned shows that “Anaferon” use may result in enhancement of interleukin-2 level. As it is known, that prescription of exogenetic interleukin-2 in HIV infected patients leads to increase in CD4 cells amount [Arduino R. C., 2004; Kovacs J. A., 2005; Levy Y., 2003; Read S. W., 2008]. Such an increase is not accompanied by the clinical response, what may be explained by the fact, that the pool of peripheral CD4 lymphocytes is refilled at the expense of the defective cells, not able to ensure a proper protective function [Rajesh T., 2009]. As it is specified in a range of researches, exogenetic interleukin-2 does not enhance the production of excitatory CD4 memory cells, what is one of the main factors of anticontagious protection. Besides, side effects of the prescribed interleukin-2 may weigh out its beneficial effect [The INSIGHT-ESPRIT Study Group and SILCAAT Scientific Committee, 2009]. We have observed that “Anaferon” use may enhance the exogenetic interleukin-2 synthesis, especially if supported by the simultaneous prescription of ARVT. A possible explanation to the aforementioned observation may lie in the fact, that “Anaferon” directly influences the effector functions of CD4 lymphocytes, what influences its functionality and enhances the interleukin-2 production. Increase in CD4 cells quantity under the influence of “Anaferon” in the researched groups may serve as an indirect proof in favor of such a consequence.

The assessment of interleukin-4 content in blood revealed a reduction in the initial level in all the groups if compared to the health group, though only groups 4 and 5 were characterized by a trustworthy twofold reduction in this cytotoxicant level. IL-4, as well as IL-2 also has a great influence on the immune response. Being involved into T-helper-2 (Th-2) immune response, it possesses if combined with interleukin-2 an antiviral effect, as it primarily influences the CD4 cells.

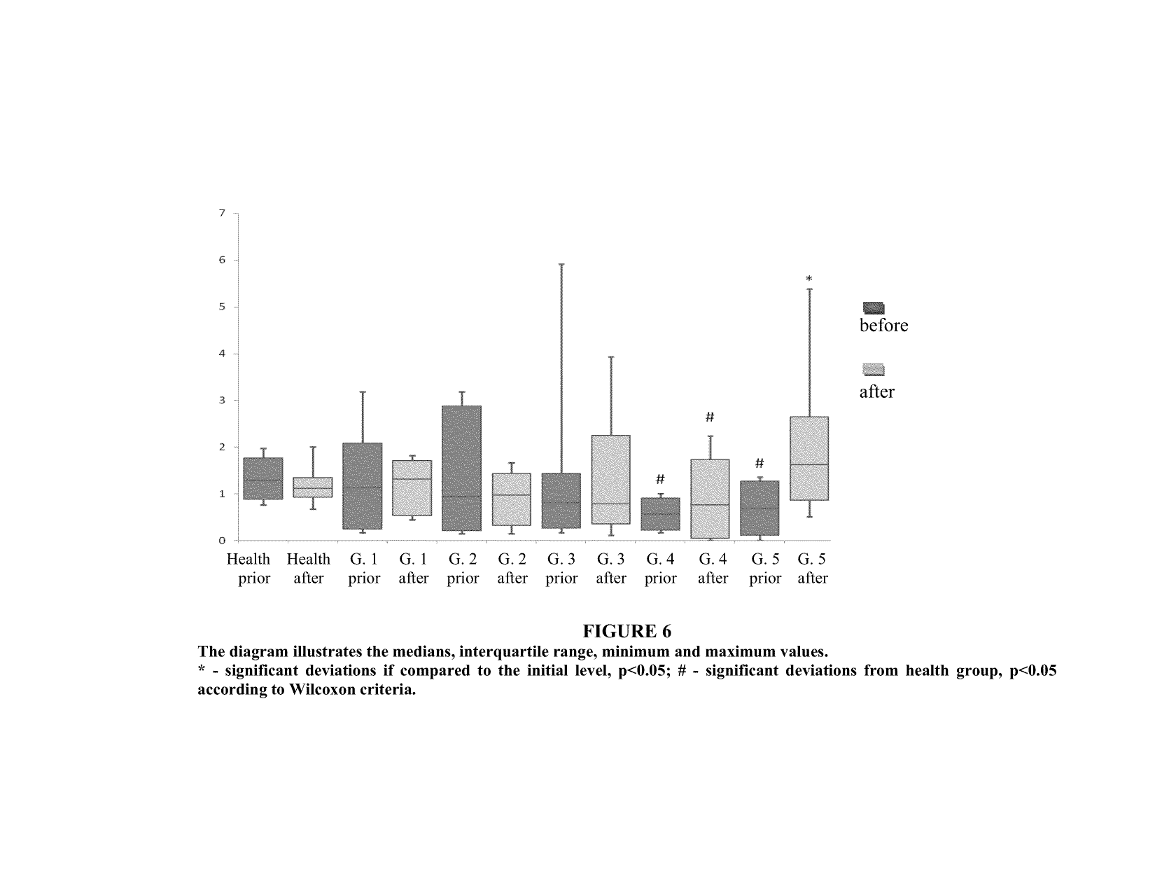

Subsequent to the use of preparation a significant increase in IL-4 level in patients of the fifth group was observed (group 5: 1.62 [0.86-2.65] cells/mcL subsequent to the course compared to 2: 0.69 [0.11-1.27] cells/mcL prior to the course, p<0.05), moreover the concentration of this cytotoxicant leveled off with such a concentration within the health group. Interleukin-4 content in blood serum of the patients, enrolled in other groups, did not reveal any observable changes (FIG. 6).

Consequently, we may conclude, that stimulation of interleukin-2 and 4 production under the influence of “Anaferon” and increase in their level has a positive effect on a general condition of such patients, as well as on opportunistic infections prevention.

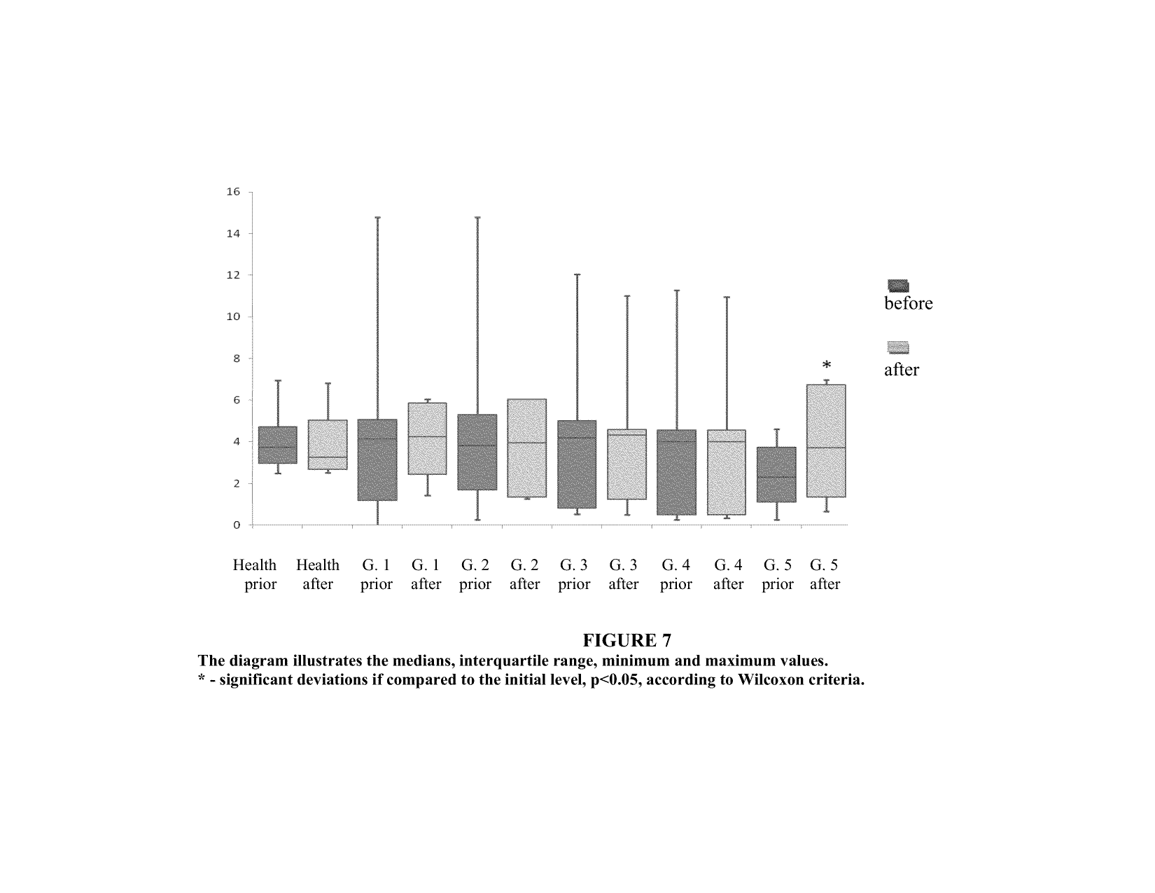

All the patients of the researched groups taken into account, only patients involved into group 5 revealed significant (p<0.05) increase in IFN-γ level (group 5: 3.71 [1.34-6.73] cells/mcL subsequent to the course compared to 2: 2.31 [1.11-3.74] cells/mcL prior to the course, p<0.05) (FIG. 7). Such an observation is consistent with the data, published in literature, which confirms, that increase in viral load and deconditioning with a transfer to the AIDS stage may be followed by an increase in IFN-γ concentration in the peripheral blood [Buch S., 2001; Eylar E. H., 2001]. Nevertheless, the results of recent researches, carried out on the human cells of monocytic and macrophage systems is an evidence of a positive influence of IFN-γ within the HIV infection, as the aforementioned cytotoxicant fosters the enhancement of macrophagocytes' microbicidal functions. Besides, from the viewpoint of contamination of non-infected macrophagocytes (population of which amounts to 50-99% in HIV infected patients) the gamma interferon produces an inhibiting effect and prevents the spread of HIV within human body. Nonetheless, active macrophagocytes are able to attract the T-lymphocytes to the nidus of infection and enhance the virus production by means of T-lymphocytes activation [Renaud M., 1999]. Therefore it is recommended to carefully monitor the “Anaferon” course in HIV infected patients at the 4B stage. As the function of gamma interferon within the HIV infection pathogenesis and its influence on the spread of virus is still understudied and the literature presents a controversial data [Rey D., 2000] it is worth recommending the monitoring of the gamma interferon concentration, as well as the degree of T-lymphocytes activation within the use of immunemodulating medications.

The index of T-helper-1 (Th-1) and T-helper-2 (Th-2) lymphocytes cytotoxicants interrelation (IFN-γ/IL-4) is often used as a more informative indicator of the T-cell immune response, rather than isolated cytotoxicants concentration. The use of the given parameter allows getting an idea of cellular or humoral immune response stimulation, as well as using it in the quality of predictor of the immune response balance displacing to the side of Th-1 and Th-2 [Belan E. B., 2006]. IFN-γ/IL-4 interrelation was altered in group 1 (3.29[3.12-5.93] subsequent to the course compared to 2: 5.73 [4.72-6.74] prior to the course) and group 2 (4.79[2.97-6.96] subsequent to the course compared to 2: 6.15 [5.04-7.12] prior to the course) to the smaller side, i.e. in groups, including patients, treated with “Anaferon” (Table 5).

The aforementioned results allow making an assumption that the use of “Anaferon” is primarily characterized by Th-1 immune response stimulation in HIV infected patients, with an exception of group of patients, suffering from 4B stage of the HIV infection.

At that, as it was mentioned previously, the use of “Anaferon” at the 4B stage of the HIV infection is associated with an increase in production of both, gamma interferon and interleukin-4, what may be an evidence of cellular or humoral immune stimulation. In this particular case the IFN-γ/IL-4 overbalance to one or the other side may indicate the shift if the immune response to the side of allergic reactions development, what would be viewed as an additional pathogenic factor, worsening the state of the disease. In such a way, the use of “Anaferon” by HIV infected patients at the 4B stage shall be considered to be a reasonable therapeutic method. At the same time, for patients at the earlier stage of HIV infection, amidst compensated immune system, the reduction in IFN-γ/IL-4 interrelation on the background of an increase in cytotoxicants concentration witnesses the humoral immune system stimulation and simultaneous preservation of cellular immune system functions.

Consequently, the use of “Anaferon” by HIV infected patients is followed by the alteration of T-lymphocytes functional activity. The monitoring of the preparation efficiency requires a dynamic assessment of interleukin-2 and 4 and gamma interferon concentration with the parallel research of the viral load.

TABLE 4
Cytotoxicants values in the control group and in HIV infected patients,
not treated with “Anaferon” prior and subsequent to the treatment course

| | | | |
|---|---|---|---|
| Index | IFN-γ, pg/ml | IL-2, pg/ml | IL-4, pg/ml |

Health

| | | | | |
|---|---|---|---|---|
| At the beginning of the | 3.74 [2.97-4.72] | 3.72 | [4.04-5.13] | 1.29 [0.89-1.77] |
| research | | | | |
| Subsequent to the | 3.25 [2.69-5.03] | 3.26 | [2.54-4.98] | 1.12 [0.93-1.35] |
| cessation of the research | | | | |

Group No. 1

| | | | | |
|---|---|---|---|---|
| before | 4.14 [1.19-5.07] | 8.37 | [6.61-10.13]# | 1.14 [0.25-2.09] |
| after | 4.25 [2.44-5.87] | 9.04 | [7.34-16.90]# | 1.31 [0.53-1.71] |

Group No. 2

| | | | | |
|---|---|---|---|---|
| before | 3.81 [1.71-5.32] | 5.95 | [3.73-9.45]# | 0.94 [0.21-2.88] |
| after | 3.96 [1.36-6.04] | 8.87 | [4.97-12.49]# | 0.97 [0.32-1.44] |

Group No. 3

| | | | | |
|---|---|---|---|---|
| At the beginning of the | 4.20 [0.83-5.01] | 10.32 | [7.78-13.64] | 0.81 [0.27-1.44] |
| research | | | | |
| Subsequent to the | 4.32 [1.24-4.58] | 11.77 | [6.23-14.00]*, # | 0.79 [0.36-2.25] |
| cessation of the research | | | | |

Group No. 4

| | | | | |
|---|---|---|---|---|
| At the beginning of the | 3.87 [0.62-4.12] | 12.11 | [11.45-15.36]# | 0.57 [0.05-1.31]# |
| research | | | | |
| Subsequent to the | 4.01 [0.49-4.56] | 11.87 | [10.99-14.85]*, # | 0.76 [0.21-0.94] |
| cessation of the research | | | | |

Group No. 5

| | | | | |
|---|---|---|---|---|
| before | 2.31 [1.11-3.74] | 6.13 | [3.02-7.82]# | 0.69 [0.11-1.27]# |
| after | 3.71 [1.34-6.73]* | 8.11 | [4.98-11.39]*, # | 1.62 [0.86-2.65]* |

*significant deviations if compared to the initial level, p < 0.05
#significant deviations from health group, p < 0.05, according to Wilcoxon criteria

TABLE 5
IFN-γ/IL-4 interrelation in examined patients prior and subsequent
to “Anaferon” course (Me [Q1-Q3])

| | At the beginning | Subsequent to the |
|---|---|---|
| Group | of the research | cessation of the research |
| Health | 2.89 [1.43-5.05] | 2.90 [1.39-4.98] |
| 1 | 5.73 [4.72-6.74] | 3.29 [3.12-5.93]* |
| 2 | 6.15 [5.04-7.12] | 4.79 [2.97-6.96]* |
| 3 | 5.18 [4.99-5.76] | 5.46 [4.83-5.69] |
| 4 | 6.78 [5.99-7.35] | 5.27 [5.08-7.44] |
| 5 | 3.70 [2.79-4.63] | 2.71 [1.17-4.02] |

*significant deviations if compared to the initial level, p < 0.05, according to Wilcoxon criteria

Influence of “Anaferon” on the life quality of the HIV infected patients. The assessment of life quality within the patients, included into the groups under research, they were offered to fill in a questionnaire, which was drafted ob the basis of the international questionnaire on general health SF-36 and adjusted to the research under review. All the scales of the questionnaire were aimed at the assessment of the physical health component (scales 1-4), psychical health component (scale 5) and a component, related to the use of medical products (scale 6). Results of the questioning are illustrated in Table 6.

TABLE 6
Life quality within the groups of patients at the beginning
of the research and subsequent to its cessation (Me [Q1-Q3])

| Groups of | | Scale | | | | | |
|---|---|---|---|---|---|---|---|
| patients | | PF (1) | P (2) | GH (3) | IR (4) | ER (5) | DI (6) |
| Health | start | 100.0 | 100.0 | 100.0 | 100.0 | 100.0 | — |
| | | [87.5- | [100.0- | [91.7- | [87.5- | [100.0- | |
| | | 100.0] | 100.0] | 100.0] | 100.0] | 100.0] | |
| | end | 100.0 | 100.0 | 100.0 | 100.0 | 100.0 | — |
| | | [87.5- | [100.0- | [91.7- | [87.5- | [100.0- | |
| | | 100.0] | 100.0] | 100.0] | 100.0] | 100.0] | |
| 1 | be- | 75.0 | 83.3 | 83.3 | 75.0 | 50.0 | — |
| | fore | [75.0- | [83.3- | [83.3- | [75.0- | [50.0- | |
| | | 100.0] | 100.0] | 100.0] | 100.0] | 100.0] | |
| | after | 75.0 | 83.3 | 83.3 | 100.0 | 100.0 | — |
| | | [75.0- | [83.3- | [83.3- | [87.5- | [50.0- | |
| | | 100.0] | 100.0] | 100.0] | 100.0] | 100.0] | |
| 2 | be- | 75.0 | 83.3 | 83.3 | 75.0 | 50.0 | 83.3 |
| | fore | [75.0- | [66.7- | [83.3- | [75.0- | [50.0- | [83.3- |
| | | 100.0] | 100.0] | 100.0] | 100.0] | 100.0] | 100.0] |
| | after | 75.0 | 83.3 | 83.3 | 100.0 | 100.0 | 100.0 |
| | | [75.0- | [79.2- | [83.3- | [75.0- | [87.5- | [83.3- |
| | | 100.0] | 100.0] | 100.0] | 100.0] | 100.0] | 100.0] |
| 3 | start | 75.0 | 83.3 | 100.0 | 75.0 | 50.0 | — |
| | | [75.0- | [83.3- | [91.7- | [75.0- | [50.0- | |
| | | 100.0] | 100.0] | 100.0] | 100.0] | 100.0] | |
| | end | 75.0 | 83.3 | 100.0 | 75.0 | 50.0 | — |
| | | [75.0- | [83.3- | [83.0- | [75.0- | [50.0- | |
| | | 100.0] | 100.0] | 100.0] | 100.0] | 100.0] | |
| 4 | start | 75.0 | 83.3 | 83.3 | 75.0 | 50.0 | 83.3 |
| | | [75.0- | [83.3- | [83.3- | [75.0- | [50.0- | [83.3- |
| | | 100.0] | 100.0] | 100.0] | 100.0] | 100.0] | 100.0] |
| | end | 75.0 | 83.3 | 83.3 | 75.0 | 50.0 | 83.3 |
| | | [75.0- | [83.3- | [83.3- | [75.0- | [50.0- | [83.3- |
| | | 100.0] | 100.0] | 100.0] | 100.0] | 100.0] | 83.3] |
| 5 | be- | 75.0 | 83.3 | 83.3 | 75.0 | 50.0 | 83.3 |
| | fore | [75.0- | [83.3- | [83.3- | [75.0- | [50.0- | [83.3- |
| | | 100.0] | 100.0] | 100.0] | 100.0] | 87.5] | 83.3] |
| | after | 75.0 | 83.3 | 83.3 | 75.0 | 50.0 | 83.3 |
| | | [75.0- | [83.3- | [83.3- | [75.0- | [50.0- | [83.3- |
| | | 100.0] | 100.0] | 100.0] | 100.0] | 87.5] | 83.3] |

Notes:
points are granted according to the adjusted scale SF-36, median, first and third quarters are taken into account. Physical functioning (PF), pain (P), general health (GH), incidence rate (IR), emotional response (ER), drugs intake (DI)
* — significant deviations if compared to the initial level, p < 0.05, according to Wilcoxon criteria

It is observed that no stable life quality enhancement is observed among HIV infected patients and healthy group representatives. Nonetheless, in group 1 (patients, treated with “Anaferon” without ARVT) and group 2 (patients, treated with “Anaferon” and ARVT) a significant increase in incidence rate (IR) shall be emphasized, what indicates a progressive step in a subjective estimation of ones own state. Moreover, group 2 reveals an increase in emotional response (ER) index.

Consequently, the use of “Anaferon” is related to the patients' life quality enhancement, what is reflected in subjective estimation of ones own state and emotional response. Our research allows us to include the domestic medical preparation “Anaferon” to the schemes of treatment and prophylaxis of acute respiratory diseases and opportunistic infections in HIV infected patients, as far as “Anaferon” significantly enhances the immune status, contributes to better life quality of the patients, not causing any adverse reactions or side effects, what allows recommending it in the quality of the immuncorrective drug at all stages of HIV infection.

What is claimed is:

1. A combination pharmaceutical composition comprising a) an activated-potentiated form of an antibody to at least one cytokine and/or an activated-potentiated form of an antibody to at least one receptor; and b) an effective amount of a nucleoside reverse transcriptase inhibitor, wherein said at least one cytokine or at least one receptor is participating in the regulation of immune process.

2. The combination pharmaceutical composition, wherein said nucleoside reverse transcriptase inhibitor is azidothymidine.

3. The combination pharmaceutical composition of claim 1, wherein the activated-potentiated form of an antibody to at least one cytokine is prepared by successive centesimal dilutions coupled with shaking of every dilution.

4. The combination pharmaceutical composition of claim 1, wherein the activated-potentiated form of an antibody to at least one receptor is prepared by successive centesimal dilutions coupled with shaking of every dilution.

5. The combination pharmaceutical composition of claim 1, wherein the activated-potentiated form of an antibody to at least one cytokine or the activated-potentiated form of an antibody to at least one receptor is in the form of a mixture of C12, C30, and C50 homeopathic dilutions or the activated-potentiated form of an antibodies to at least one receptor is impregnated onto a solid carrier.

6. The combination pharmaceutical composition of claim 1, wherein said antibody is a monoclonal, polyclonal or natural antibody.

7. The combination pharmaceutical composition of claim 6, wherein said carrier is impregnated with a mixture of said dilutions.

8. The combination pharmaceutical composition of claim 2, wherein said at least one cytokine is gamma interferon.

9. The combination pharmaceutical composition of claim 8, which comprises active ingredients consisting essentially of said activated-potentiated form of antibody to gamma interferon and said effective amount of azidothymidine.

10. The combination pharmaceutical composition of claim 2, wherein said at least one receptor is CD4 receptor.

11. The combination pharmaceutical composition of claim 10, which comprises active ingredients consisting essentially of said activated-potentiated form of antibody to CD4 and said effective amount of azidothymidine.

12. The combination pharmaceutical composition of claim 2, wherein said at least one cytokine is gamma interferon and alpha interferon.

13. The combination pharmaceutical composition of claim 12, which comprises active ingredients consisting essentially of said activated-potentiated forms of antibody to gamma interferon and alpha interferon and said effective amount of azidothymidine.

14. The combination pharmaceutical composition of claim 2, wherein said at least one receptor is CD4 receptor and CD8 receptor.

15. The combination pharmaceutical composition of claim 14, which comprises active ingredients consisting essentially of said activated-potentiated forms of antibody to CD4 receptor and CD8 receptor, and said effective amount of azidothymidine.

16. The combination pharmaceutical composition of claim 1, wherein said activated-potentiated form is obtained by multiple consecutive dilutions of an initial matrix solution of said at least one antibody having the concentration ranging from 0.5 mg/ml to 5.0 mg/ml.

17. A method of treating viral infectious disease, said method comprising administering to a patient in need thereof the pharmaceutical composition of claims 1-16.

18. The method of claim 17, wherein said viral infectious disease is a disease or condition caused by HIV or associated with HIV.

19. The method of claim 18, wherein said disease and condition caused by HIV or associated with HIV is AIDS.

20. A method of HIV prophylaxis, said method comprising administering to a patient in need thereof the pharmaceutical composition of claims 1-16.

21. The pharmaceutical composition of claim 1, which is a solid oral dosage form.

22. The pharmaceutical composition of claim 22, wherein said solid oral dosage form is a tablet.
